(19) United States
(12) Patent Application Publication
(10) Pub. No.: US 2025/0277204 A1
(43) Pub. Date: Sep. 4, 2025

(54) INSECT-DERIVED ASPARTATE DECARBOXYLASES AND VARIANTS THEREOF FOR IMPROVED BETA-ALANINE PRODUCTION

(71) Applicant: MOJIA BIOTECH LTD., Shanghai (CN)

(72) Inventors: Man Kit LAU, Shanghai (CN); Xu LOU, Shanghai (CN); Jinhuan SU, Shanghai (CN); Congming ZENG, Shanghai (CN); Tailong JIANG, Shanghai (CN); Ansen CHIEW, Shanghai (CN)

(73) Assignee: MOJIA BIOTECH LTD., Shanghai (CN)

(21) Appl. No.: 18/548,325

(22) PCT Filed: Mar. 3, 2022
(86) PCT No.: PCT/CN2022/079042

§ 371 Date: Aug. 29, 2023

(30) Foreign Application Priority Data
Mar. 3, 2021 (WO) PCT/CN2021/078949

(51) Int. Cl. C12N 9/88; C12P 13/06

(52) U.S. Cl. CPC C12N 9/88; C12P 13/06; C12Y 401/01011

(57) ABSTRACT

Provided are N-terminally truncated variants of insect aspartate 1-decarboxylases that exhibit improved performance for beta-alanine production.

### CROSS-REFERENCE TO RELATED APPLICATIONS

This application is the U.S. National Stage of International Application No. PCT/CN2022/079042, filed on Mar. 3, 2022, which designates the U.S., published in English, and claims the priority benefit of International Application No. PCT/CN2021/078949, filed on Mar. 3, 2021.

The present description relates to biological processes for the production of beta-alanine. More particularly, described herein are insect aspartate 1-decarboxylase (ADC) enzymes and variants thereof that are particularly advantageous for beta-alanine production from L-aspartate.

### BACKGROUND

Beta-alanine, also known as beta-aminopropionic acid or 3-aminopropionic acid, is a naturally occurring amino acid in which the amino group is at the beta-position from the carboxylate group. Beta-alanine is a multi-purpose organic synthetic raw material, mainly used to synthesize pantothenic acid and calcium pantothenate, carnosine, pamidronate, balsalazide, etc. It is widely used in medicine, feed, food and other fields, and has a large market demand. At industrial scales, beta-alanine is currently produced by chemical processes involving harsh reaction conditions with safety concerns, high equipment costs, and environmental pollution. The production of beta-alanine by safer and more environmentally friendly biological processes have been greatly hampered by enzymes having poor activity, expression, and/or stability, thereby making such processes not commercially viable in comparison to chemical synthesis approaches. Therefore, improved enzymes useful for the biological production of beta-alanine are highly desirable.

### SUMMARY

In one aspect, described herein is a recombinant truncated insect aspartate 1-decarboxylase (ADC), the truncated insect ADC lacking a sufficient number of contiguous residues within the amino terminal region of a corresponding full length wild-type insect ADC such that the truncated ADC exhibits increased conversion of aspartate to beta-alanine as compared to the corresponding full length wild-type insect ADC.

In a further aspect, described herein is a recombinant protein having aspartate 1-decarboxylase activity, the recombinant protein comprising an amino acid sequence at least 80, 81, 82, 83, 84, 85, 86, 87, 88, 89, 90, 91, 92, 93, 94, 95, 96, 97, 98, or 99% identical overall to:
(a) position 72 to 561 of the amino acid sequence of CtADC set forth in SEQ ID NO: 2;
(b) position 79 to 568 of the amino acid sequence of AaADC set forth in SEQ ID NO: 4;
(c) position 56 to 544 of the amino acid sequence of AtADC set forth in SEQ ID NO: 3;
(d) position 52 to 540 of the amino acid sequence of TcADC set forth in SEQ ID NO: 1;
(e) position 71 to 560 of the amino acid sequence of Aa2ADC set forth in SEQ ID NO: 10;
(f) position 71 to 562 of the amino acid sequence of Aa3ADC set forth in SEQ ID NO: 11;
(g) position 74 to 563 of the amino acid sequence of CqADC set forth in SEQ ID NO: 9;
(h) position 72 to 561 of the amino acid sequence of Aa4ADC set forth in SEQ ID NO: 13;
(i) position 74 to 624 of the amino acid sequence of AdADC set forth in SEQ ID NO: 14;
(j) position 83 to 572 of the amino acid sequence of AsADC set forth in SEQ ID NO: 12;
(k) position 72 to 561 of the amino acid sequence of As2ADC set forth in SEQ ID NO: 15;
(1) position 53 to 541 of the amino acid sequence of TmADC set forth in SEQ ID NO: 6;
(m) position 57 to 572 of the amino acid sequence of AvADC set forth in SEQ ID NO: 5; or
(n) position 1 to 491 of the amino acid sequence of SEQ ID NO: 29.

In a further aspect, described herein is a polynucleotide comprising a nucleic acid sequence encoding the recombinant truncated insect ADC described herein, or the recombinant protein described herein.

In a further aspect, described herein is an expression cassette comprising the isolated or recombinant polynucleotide described herein operably linked to a promoter that is heterologous with respect to the insect ADC.

In a further aspect, described herein is a host cell that expresses the recombinant truncated insect ADC described herein, the recombinant protein described herein, and/or is transformed with or engineered to comprise the polynucleotide described herein or the expression cassette described herein.

In a further aspect, described herein is a process for the production of beta-alanine, the process comprising: (a) providing an ADC enzyme source which is the truncated insect ADC described herein, the recombinant protein described herein, and/or the host cell described herein; (b) contacting the ADC enzyme source with a source of aspartate under conditions enabling the ADC enzyme source to catalyze the conversion of the aspartate to beta-alanine; and (c) isolating and/or concentrating the beta-alanine produced.

In a further aspect, described herein is a composition comprising beta-alanine produced by the process described herein.

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one” but it is also consistent with the meaning of “one or more”, “at least one”, and “one or more than one”.

The term “about” is used to indicate that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

As used herein, the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or process/method steps.

As used herein, the term “beta-alanine” includes beta-alanine as well as beta-alanine salts (e.g., calcium, sodium, or potassium beta-alanine salt).

### BRIEF DESCRIPTION OF THE DRAWINGS

In the appended drawings:

FIG. 1 shows a phylogenetic tree of ADC enzymes from different insect species grouped by 85% sequence identity. Activity data for some ADCs tested in Example 2 are shown.

FIG. 2 shows an alignment of amino acid sequences of ADCs identified from nine different mosquito species. The broken line delineates a region in the N terminal portion that is poorly conserved across mosquito ADCs. A glycine residue at position 96 that is unique to CtADC is highlighted in black. SEQ IN NOS corresponding to each sequence are in parentheses.

FIG. 3 shows an alignment the N-terminal amino acid sequences of mosquito and beetle ADCs described in Examples 5 and 6. N-terminal truncations between the two residues highlighted in black resulted in a truncated ADC having increased activity as compared to its corresponding full-length protein, while N-terminal truncations between the two residues boxed in white resulted in enzymes with low or no detectable ADC activity. The region indicated with a broken line delineates the position where N-terminal truncations may be expected to cease being beneficial for enzymatic activity.

### SEQUENCE LISTING

This application contains a Sequence Listing which has been submitted electronically in computer readable form (ASCII text format) created on Apr. 8, 2024, is named “07 ROBCNZ00700_20240408_sequence_listing.txt”, and is 126,187 bytes in size, The computer readable form is incorporated herein by reference.

TABLE 1
Sequence Listing Description

| SEQ ID NO: | Description |
|---|---|
| 1 | Tribolium castaneum (Tc) WT ADC |
| 2 | Culex tarsalis (Ct) WT ADC |
| 3 | Aethina tumida (At) WT ADC |
| 4 | Anopheles arabiensis (Aa) WT ADC |
| 5 | Asbolus verrucosus (Av) WT ADC |
| 6 | Tenebrio molitor (Tm) WT ADC |
| 7 | Cryptotermes secundus (Cs) WT ADC |
| 8 | Drosophila melanogaster (Dm) WT ADC |
| 9 | Culex quinquefasciatus (Cq) WT ADC |
| 10 | Aedes albopictus (Aa2) WT ADC |
| 11 | Aedes aegypti (Aa3) WT ADC |
| 12 | Anopheles sinensis (As) WT ADC |
| 13 | Anopheles albimanus (Aa4) WT ADC |
| 14 | Anopheles darlingi (Ad) WT ADC |
| 15 | Anopheles stephensi (As2) WT ADC |
| 16 | Tribolium castaneum (Tc) ADC WT cDNA |
| 17 | Culex tarsalis (Ct) ADC WT cDNA |
| 18 | Aethina tumida (At) ADC WT cDNA |
| 19 | Anopheles arabiensis (Aa) ADC WT cDNA |
| 20 | Asbolus verrucosus (Av) ADC WT cDNA |
| 21 | Tenebrio molitor (Tm) ADC WT cDNA |
| 22 | Cryptotermes secundus (Cs) ADC WT cDNA |
| 23 | Drosophila melanogaster (Dm) ADC WT cDNA |
| 24 | Culex quinquefasciatus (Cq) ADC WT cDNA |
| 25 | Aedes albopictus (Aa2) ADC WT cDNA |
| 26 | Aedes aegypti (Aa3) ADC WT cDNA |
| 27 | Anopheles sinensis (As) ADC WT cDNA |
| 28 | Conserved N-term 15-aa mosquito ADC sequence |
| 29 | Combined ADC WT sequences sharing greater than |
| | 70% identity with N-terminal truncated CtADC |

### DETAILED DESCRIPTION

Attempts at industrial-scale biological synthesis of beta-alanine via the enzyme-catalyzed removal of the alpha carboxy group of L-aspartate has been greatly impeded by enzymes having poor activity, expression, and/or stability, thereby making such processes not commercially viable in comparison to chemical synthesis approaches. Improved enzymes catalyzing the conversion of L-aspartate to beta-alanine having increased activity, expression, and/or stability would greatly facilitate the biological synthesis of beta-alanine on a commercial scale. The present description relates to the discovery that certain insect-derived enzymes having aspartate 1-decarboxylase activity are particularly advantageous for beta-alanine production, and further that the performance of such insect-derived enzymes may be greatly improved by truncating portions of their N terminus.

In a first aspect, described herein are recombinant truncated insect aspartate 1-decarboxylase (ADC) enzymes that are particularly advantageous for beta-alanine production. As used herein, the expression “aspartate 1-decarboxylase” or “ADC” refers to a polypeptide having the ability to catalyze the enzymatic conversion of L-aspartate to beta-alanine and carbon dioxide. In some embodiments, such polypeptides may include those categorized under the enzyme class E.C. 4.1.1.11. In some embodiments, such polypeptides may also include enzymes categorized in other enzyme classes (e.g., enzymes also having activity on substates other than L-aspartate) and/or polypeptides that may have been annotated (e.g., in public databases) as enzymes other than an ADC (e.g., glutamate decarboxylase, cysteine sulfinic acid decarboxylase). In some embodiments, insect ADCs and truncated variants thereof described herein may include enzymes that have both aspartate 1-decarboxylase activity and cysteine sulfinic acid decarboxylase activity.

As used herein, the term “truncated” or “truncation” includes not only removal of a segment of a protein starting from a terminal residue (e.g., starting from an N-terminal methionine of a recombinant protein) but may also include deletions of contiguous residues within a terminal region or portion of a protein (e.g., wild-type protein) such that the terminal portion of the truncated protein is shorter than the that of the untruncated protein.

In some embodiments, the truncated insect ADCs described herein lack a sufficient number of contiguous residues within the amino terminal portion of their corresponding full length wild-type insect ADCs such that the truncated ADC exhibits increased conversion of aspartate to beta-alanine as compared to their parent full length wild-type proteins. In some embodiments, increased conversion of aspartate to beta-alanine may include increased ADC catalytic activity, increased ADC stability, and/or increased expression relative to the corresponding full length wild-type protein.

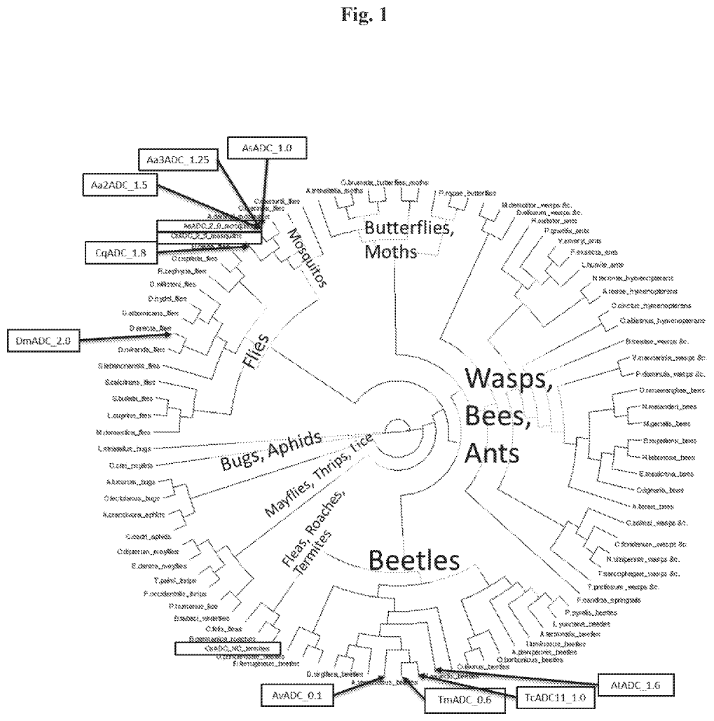

In some embodiments, truncated ADCs described herein may be a truncated variant of an organism of the Class Insecta (e.g., a mosquito, fly, beetle, flea, roach, or termite ADC). In particular embodiments, truncated insect ADCs described herein may be a truncated variant of a mosquito, fly, or beetle ADC for which structural relationships are shown in the phylogenetic tree of FIG. 1. In some embodiments, truncated insect ADCs described herein may be a truncated variant of an insect ADC from the genus: Culex, Anopheles, Drosophila, Aethina, Aedes, Tribolium, Anopheles, Tenebrio, Asbolus, or Cryptotermes. In some embodiments, truncated insect ADCs described herein may include a truncated variant of an insect ADC from the species: Culex tarsalis, Anopheles arabiensis, Drosophila melanogaster, Culex quinquefasciatus, Aethina tumida, Aedes albopictus, Aedes aegypti, Tribolium castaneum, Anopheles sinensis, Tenebrio molitor, Asbolus verrucosus, or Cryptotermes secundus. In some embodiments, the truncated insect ADCs described herein may be a truncated variant of a wild-type ADC enzyme, the wild-type ADC enzyme being defined by an amino acid sequence at least 90, 91, 92, 93, 94, 95, 96, 97, 98, or 99% identical overall to the amino acid sequence of SEQ ID NO: 29.

In some embodiments, truncated ADCs described herein may be a truncated variant of a mosquito ADC comprising an amino acid sequence at least 80, 81, 82, 83, 84, 85, 86, 87, 88, 89, 90, 91, 92, 93, 94, 95, 96, 97, 98, or 99% identical overall to any one of SEQ ID NOs: 2, 4, or 9-15. In some embodiments, truncated ADCs described herein may be a truncated variant of a beetle ADC comprising an amino acid sequence at least 80, 81, 82, 83, 84, 85, 86, 87, 88, 89, 90, 91, 92, 93, 94, 95, 96, 97, 98, or 99% identical overall to any one of SEQ ID NOs: 1, 3, or 5-6. In some embodiments, truncated ADCs described herein may be a truncated variant of a fly ADC comprising an amino acid sequence at least 80, 81, 82, 83, 84, 85, 86, 87, 88, 89, 90, 91, 92, 93, 94, 95, 96, 97, 98, or 99% identical overall to SEQ ID NO: 8.

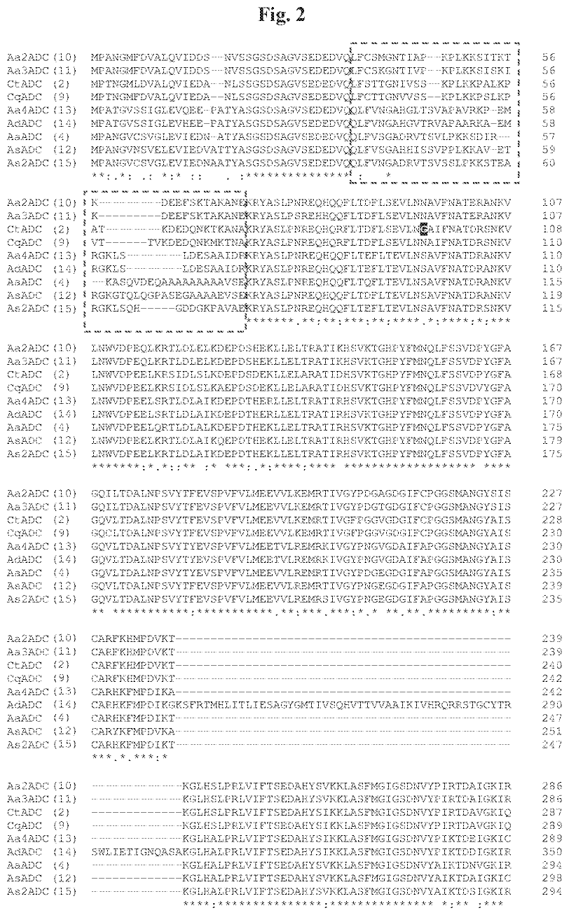

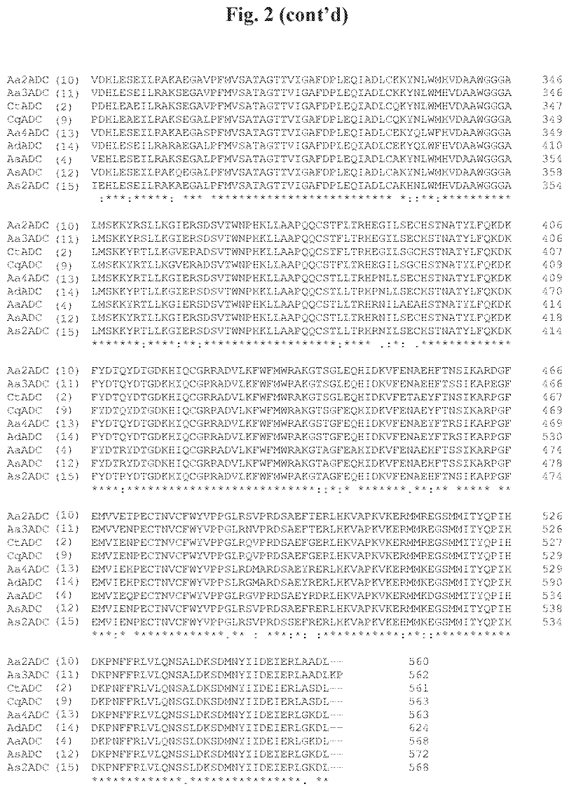

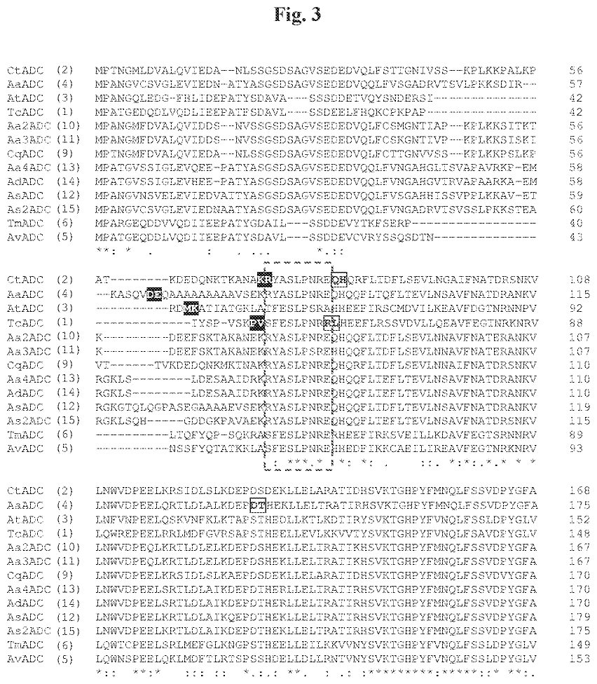

In some embodiments, truncated ADCs described herein may comprise an amino acid sequence at least 80, 81, 82, 83, 84, 85, 86, 87, 88, 89, 90, 91, 92, 93, 94, 95, 96, 97, 98, or 99% identical overall to an N-terminally truncated fragment of an ADC that exhibits increased activity relative to its untruncated (e.g., full-length) parent enzyme. In some embodiments, truncated ADCs described herein may comprise an amino acid sequence at least 80, 81, 82, 83, 84, 85, 86, 87, 88, 89, 90, 91, 92, 93, 94, 95, 96, 97, 98, or 99% identical overall to: (a) position 72 to 561 of the amino acid sequence of CtADC set forth in SEQ ID NO: 2; (b) position 79 to 568 of the amino acid sequence of AaADC set forth in SEQ ID NO: 4; (c) position 56 to 544 of the amino acid sequence of AtADC set forth in SEQ ID NO: 3; (d) position 52 to 540 of the amino acid sequence of TcADC set forth in SEQ ID NO: 1; (e) position 71 to 560 of the amino acid sequence of Aa2ADC set forth in SEQ ID NO: 10; (f) position 71 to 562 of the amino acid sequence of Aa3ADC set forth in SEQ ID NO: 11; (g) position 74 to 563 of the amino acid sequence of CqADC set forth in SEQ ID NO: 9; (h) position 72 to 561 of the amino acid sequence of Aa4ADC set forth in SEQ ID NO: 13; (i) position 74 to 624 of the amino acid sequence of AdADC set forth in SEQ ID NO: 14; (j) position 83 to 572 of the amino acid sequence of AsADC set forth in SEQ ID NO: 12; (k) position 72 to 561 of the amino acid sequence of As2ADC set forth in SEQ ID NO: 15; (1) position 53 to 541 of the amino acid sequence of TmADC set forth in SEQ ID NO: 6; (m) position 57 to 572 of the amino acid sequence of AvADC set forth in SEQ ID NO: 5; or (n) position 1 to 491 the amino acid sequence set forth in SEQ ID NO: 29. These segments correspond to fragments of wild-type full length insect ADCs that either are shown to exhibit increased performance in beta-alanine production or may be expected to do so based on sequence conservation and multiple sequence alignments such as in Examples 5-7 and in FIGS. 2 and 3.

In some embodiments, truncated ADCs described herein may lack at least X contiguous residues of the amino terminus of the corresponding full length wild-type insect ADC, wherein X is any integer between 5 and 50. In some embodiments, truncated ADCs described herein may lack at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 contiguous residues of the amino terminus of the corresponding full length wild-type insect ADC, depending on the length of the amino terminus of the corresponding full length wild-type insect ADC.

In some embodiments, truncated ADCs described herein may be truncated at a position immediately C-terminal (downstream) of a residue corresponding to position n of a full-length wild-type insect ADC, wherein n is any integer between 2 and Y, wherein Y is the most C-terminal residue position within the full-length wild-type insect ADC at which truncation can occur with the truncated ADC exhibiting increased conversion of aspartate to beta-alanine as compared to the full length wild-type ADC. As used herein in the context of amino acid residue numbering, the expression “corresponding to position” considers that amino acid residue numbering differs amongst different proteins (e.g., different insect ADCs) but that a person of skill in the art would be able to determine corresponding residue positions in two proteins sharing a degree of amino acid sequence identity by performing sequence alignments between the two proteins, optionally including additional orthologs to identify conserved residues using widely available softwares (e.g., Clustal Omega) as demonstrated herein.

In some embodiments, truncated ADCs described herein may be truncated at a position C-terminal (downstream) of a residue corresponding to any one of: (a) position 2, 3, 4, 5, 6, 7, 8, 9 10, or 11 to 71 of the amino acid sequence of CtADC set forth in SEQ ID NO: 2; (b) position 2, 3, 4, 5, 6, 7, 8, 9 10, or 11 to 78 of the amino acid sequence of AaADC set forth in SEQ ID NO: 4; (c) position 2, 3, 4, 5, 6, 7, 8, 9 10, or 11 to 55 of the amino acid sequence of AtADC set forth in SEQ ID NO: 3; (d) position 2, 3, 4, 5, 6, 7, 8, 9 10, or 11 to 51 of the amino acid sequence of TcADC set forth in SEQ ID NO: 1; (e) position 2, 3, 4, 5, 6, 7, 8, 9 10, or 11 to 70 of the amino acid sequence of Aa2ADC set forth in SEQ ID NO: 10; (f) position 2, 3, 4, 5, 6, 7, 8, 9 10, or 11 to 70 of the amino acid sequence of Aa3ADC set forth in SEQ ID NO: 11; (g) position 2, 3, 4, 5, 6, 7, 8, 9 10, or 11 to 73 of the amino acid sequence of CqADC set forth in SEQ ID NO: 9; (h) position 2, 3, 4, 5, 6, 7, 8, 9 10, or 11 to 73 of the amino acid sequence of Aa4ADC set forth in SEQ ID NO: 13; (i) position 2, 3, 4, 5, 6, 7, 8, 9 10, or 11 to 73 of the amino acid sequence of AdADC set forth in SEQ ID NO: 14; (j) position 2, 3, 4, 5, 6, 7, 8, 9 10, or 11 to 82 of the amino acid sequence of AsADC set forth in SEQ ID NO: 12; (k) position 2, 3, 4, 5, 6, 7, 8, 9 10, or 11 to 78 of the amino acid sequence of As2ADC set forth in SEQ ID NO: 15; (1) position 2, 3, 4, 5, 6, 7, 8, 9 10, or 11 to 52 of the amino acid sequence of TmADC set forth in SEQ ID NO: 6; (m) position 2, 3, 4, 5, 6, 7, 8, 9 10, or 11 to 56 of the amino acid sequence of AvADC set forth in SEQ ID NO: 5; or (n) position 1 to 491 of the amino acid consensus sequence of SEQ ID NO: 29. The upper limit of each of the amino acid sequences mentioned above refers to the residue positions within the full length wild-type insect ADCs shown in Table 7 and FIG. 3 and corresponding to K71 of CtADC. N-terminal truncations at least up to K71 of CtADC resulted in a truncated ADC (CtADC72-561) exhibiting increased conversion of aspartate to beta-alanine as compared to the full length wild-type ADC.

In some embodiments, truncated ADCs described herein may be truncated at a position N-terminal (upstream) of a residue corresponding to any one of: (a) position 72 to 80 of the amino acid sequence of CtADC set forth in SEQ ID NO: 2; (b) position 79 to 87 of the amino acid sequence of AaADC set forth in SEQ ID NO: 4; (c) position 56 to 64 of the amino acid sequence of AtADC set forth in SEQ ID NO: 3; (d) position 52 to 60 of the amino acid sequence of TcADC set forth in SEQ ID NO: 1; (e) position 71 to 79 of the amino acid sequence of Aa2ADC set forth in SEQ ID NO: 10; (f) position 71 to 79 of the amino acid sequence of Aa3ADC set forth in SEQ ID NO: 11; (g) position 74 to 82 of the amino acid sequence of CqADC set forth in SEQ ID NO: 9; (h) position 72 to 82 of the amino acid sequence of Aa4ADC set forth in SEQ ID NO: 13; (i) position 74 to 82 of the amino acid sequence of AdADC set forth in SEQ ID NO: 14; (j) position 83 to 91 of the amino acid sequence of AsADC set forth in SEQ ID NO: 12; (k) position 72 to 87 of the amino acid sequence of As2ADC set forth in SEQ ID NO: 15; (1) position 53 to 61 of the amino acid sequence of TmADC set forth in SEQ ID NO: 6; (m) position 57 to 65 of the amino acid sequence of AvADC set forth in SEQ ID NO: 5; or (n) position 1 to 491 the amino acid sequence set forth in SEQ ID NO: 29. These residue positions correspond to those delineated in Table 7 and by a broken line in FIG. 3.

In some embodiments, truncated ADCs described herein may be truncated at a position N-terminal (upstream) of a residue corresponding to any one of: (a) position 75 of the amino acid sequence of CtADC set forth in SEQ ID NO: 2; (b) position 82 of the amino acid sequence of AaADC set forth in SEQ ID NO: 4; (c) position 59 of the amino acid sequence of AtADC set forth in SEQ ID NO: 3; (d) position 55 of the amino acid sequence of TcADC set forth in SEQ ID NO: 1; (e) position 74 of the amino acid sequence of Aa2ADC set forth in SEQ ID NO: 10; (f) position 74 of the amino acid sequence of Aa3ADC set forth in SEQ ID NO: 11; (g) position 77 of the amino acid sequence of CqADC set forth in SEQ ID NO: 9; (h) position 77 of the amino acid sequence of Aa4ADC set forth in SEQ ID NO: 13; (i) position 77 of the amino acid sequence of AdADC set forth in SEQ ID NO: 14; (j) position 86 of the amino acid sequence of AsADC set forth in SEQ ID NO: 12; (k) position 82 of the amino acid sequence of As2ADC set forth in SEQ ID NO: 15; (1) position 56 of the amino acid sequence of TmADC set forth in SEQ ID NO: 6; (m) position 60 of the amino acid sequence of AvADC set forth in SEQ ID NO: 5; or (n) position 1 to 491 the amino acid sequence set forth in SEQ ID NO: 29. These residue positions correspond to S75 in CtADC that occurs within a tripeptide sequence “SLP” being conversed across all the insect sequences aligned in FIG. 3.

In a further aspect, described herein are recombinant proteins having aspartate 1-decarboxylase activity, the recombinant proteins comprising an amino acid sequence at least 80, 81, 82, 83, 84, 85, 86, 87, 88, 89, 90, 91, 92, 93, 94, 95, 96, 97, 98, or 99% identical overall to: (a) position 72 to 561 of the amino acid sequence of CtADC set forth in SEQ ID NO: 2; (b) position 79 to 568 of the amino acid sequence of AaADC set forth in SEQ ID NO: 4; (c) position 56 to 544 of the amino acid sequence of AtADC set forth in SEQ ID NO: 3; (d) position 52 to 540 of the amino acid sequence of TcADC set forth in SEQ ID NO: 1; (e) position 71 to 560 of the amino acid sequence of Aa2ADC set forth in SEQ ID NO: 10; (f) position 71 to 562 of the amino acid sequence of Aa3ADC set forth in SEQ ID NO: 11; (g) position 74 to 563 of the amino acid sequence of CqADC set forth in SEQ ID NO: 9; (h) position 72 to 561 of the amino acid sequence of Aa4ADC set forth in SEQ ID NO: 13; (i) position 74 to 624 of the amino acid sequence of AdADC set forth in SEQ ID NO: 14; (j) position 83 to 572 of the amino acid sequence of AsADC set forth in SEQ ID NO: 12; (k) position 72 to 561 of the amino acid sequence of As2ADC set forth in SEQ ID NO: 15; (1) position 53 to 541 of the amino acid sequence of TmADC set forth in SEQ ID NO: 6; (m) position 57 to 572 of the amino acid sequence of AvADC set forth in SEQ ID NO: 5; or (n) position 1 to 491 the amino acid sequence set forth in SEQ ID NO: 29. These regions were not only found herein to be highly conserved amongst at least mosquito and beetle ADCs, but they also correspond to a truncated variant of CtADC72-561 which was found to exhibit increased conversion of aspartate to beta-alanine as compared to the full length wild-type CtADC.

In some embodiments, truncated ADCs and/or recombinant proteins described herein may comprise a glycine residue at a position corresponding to position 96 of the amino acid sequence of CtADC set forth in SEQ ID NO: 2. CtADC outperformed other insect-derived ADCs by a significant margin from the enzyme activity testing performed in Example 2, including a 39% increase in activity over counterpart mosquito enzyme CqADC with which it shares about 97% amino acid sequence identity. A comparison of amino acid differences between CtADC and CqADC in the catalytic portion of the enzymes performed in Example 8 revealed a single glycine residue at position 96 of CtADC that was unique amongst all other insect sequences analyzed (see FIG. 3), suggesting that this residue may play a role in the heightened beta-alanine production associated with CtADC.

In a further aspect, described herein is a polynucleotide comprising a nucleic acid sequence encoding a recombinant truncated insect ADC or a recombinant protein as described herein. In some embodiments, the polynucleotide is DNA. In some embodiments, the polynucleotide is RNA.

In a further aspect, described herein is a polynucleotide molecule that hybridizes under stringent conditions to the full complement of the nucleic acid sequence of any one of SEQ ID NOs: 16 to 27; a nucleic acid sequence encoding the recombinant truncated insect ADC as described herein; a nucleic acid sequence encoding the truncated recombinant protein as described herein; or any combination thereof.

In a further aspect, described herein is an expression cassette comprising the isolated or recombinant polynucleotide described herein operably linked to a promoter (e.g., that is heterologous with respect to the insect ADC).

In a further aspect, described herein is a host cell that expresses a recombinant truncated insect ADC or a recombinant protein as described herein, and/or is transformed with or engineered to comprise a polynucleotide or expression cassette described herein. In some embodiments, the host cell may be a microbial cell. In some embodiments, the host cell may be a bacterial, insect, mammalian, yeast or fungal cell.

In a further aspect, a recombinant truncated insect ADC, a recombinant protein, or a host cell described herein, may be for use in the industrial production of beta-alanine from aspartate. In a further aspect, described herein is a process for the production of beta-alanine, the process comprising: (a) providing an ADC enzyme source which is a truncated insect ADC as described herein, a recombinant protein as described herein, and/or a host cell as described herein; (b) contacting the ADC enzyme source with a source of aspartate under conditions enabling the enzyme source to catalyze the conversion of the aspartate to beta-alanine; and (c) isolating and/or concentrating the beta-alanine produced. In some embodiments, the host cells expressing the recombinant truncated insect ADC or the recombinant protein described herein may be utilized as intact cells, which advantageously may prevent cellular debris from lysed cells from contaminating the beta-alanine produced.

In a further aspect, described herein is a composition comprising beta-alanine produced by a process described herein.

### EXAMPLES

### Example 1: General Materials and Methods

### Cloning and Expression of L-Aspartate-α-Decarboxylase (ADC) Enzymes

Codon optimized cDNA sequences of the ADCs that were cloned and expressed in bacteria are shown in SEQ ID NOs: 16-27. The cDNA sequences of ADCs were cloned into separate expression vectors and transformed into Escherichia coli to enhance the expression of ADCs upon addition of an inducer. For N-terminal truncations, the desired number of amino acids downstream of the initiator methionine were deleted.

### ADC Activity Measurements

ADC activity was measured by first, growing BL21 (DE3) E. coli cells expressing the ADC of interest in 500 μL LB broth containing kanamycin and 0.2% isopropyl β-d-1-thiogalactopyranoside (IPTG) for 24 hours at 30° C. Cells were then pelleted to remove the supernatant, resuspended and ultrasonic-crushed. The plate was then centrifuged to remove any debris, and the supernatant containing the cell lysates was collected. The cell lysates containing the ADCs were then tested for activity by incubating 50 μL of the supernatant in a 50 mL solution containing L-aspartic acid with a final concentration of 60 g/L and pyridoxal phosphate (PLP) with a final concentration of 0.2 g/L at a pH of 6.5, a temperature of 37° C., and stirred at 200 rpm. 1M of sulfuric acid was then titrated into the reaction solution to maintain the pH. After one hour, the amount of sulfuric acid used by the reaction was determined to directly measure ADC activity. Experiments were conducted at least 3 times, and the average activity value was calculated.

### Example 2: Activity of Insect-Derived ADCs

A large-scale screening was performed to compare the expression and activity of ADC enzymes from a plurality of different prokaryotic and eukaryotic organisms when recombinantly expressed in bacterial host cells. The screening revealed that lysates of bacterial cells transformed with ADCs from insect species exhibited consistently higher beta-alanine production than ADCs from other organisms. Table 2 shows relative ADC activities of lysates from bacteria transformed with codon-optimized cDNAs of ADCs from mosquito, fly and beetle species, measured as described in Example 1. Interestingly, lysate from bacteria transformed with an ADC from the mosquito species Culex tarsalis (CtADC; SEQ ID NO: 2) significantly outperformed all other enzymes tested.

TABLE 2
Activity of ADCs

| Enzyme | SEQ ID NO: | Activity | Insect | Species |
|---|---|---|---|---|
| CtADC | 2 | 2.5 | mosquito | Culex tarsalis |
| AaADC | 4 | 2.0 | mosquito | Anopheles arabiensis |
| DmADC | 8 | 2.0 | fly | Drosophila melanogaster |
| CqADC | 9 | 1.8 | mosquito | Culex quinquefasciatus |
| AtADC | 3 | 1.6 | beetle | Aethina tumida |
| Aa2ADC | 10 | 1.5 | mosquito | Aedes albopictus |
| Aa3ADC | 11 | 1.25 | mosquito | Aedes aegypti |
| TcADC | 1 | 0.7 | beetle | Tribolium castaneum |
| AsADC | 12 | 1.0 | mosquito | Anopheles sinensis |
| TmADC | 6 | 0.6 | beetle | Tenebrio molitor |
| AvADC | 5 | 0.1 | beetle | Asbolus verrucosus |

### Example 3: Insect-Derived ADC Sequence Analysis

The amino acid sequence of CtADC was used as the basis of a Protein BLAST™ to identify other ADCs from various species. Over 5000 hits sequences were retrieved, of which 188 with the highest BLAST score sequences were then selected, combined with the sequences of the insect-derived ADCs of Table 2, grouped by 85% sequence identity, and finally incorporated in a broad insect phylogenetic tree (FIG. 1). The phylogenetic tree shown in FIG. 1 shows that mosquito and fly ADCs are structurally related, and that the beetle, flea, roach and termite ADCs are structurally related.

### Example 4: Mosquito-Derived ADC Sequence Analysis

An alignment of amino acid sequences of ADCs identified from nine different mosquito species was performed with Clustal Omega (1.2.4) and is shown in FIG. 2. The alignment revealed relatively high sequence conservation throughout different mosquito species, as shown below in the percent identity matrix in Table 3.

TABLE 3
Percent identity matrix of mosquito-derived ADCs

| | Aa2ADC | Aa3ADC | CtADC | CqADC | Aa4ADC | AdADC | AaADC | AsADC | As2ADC |
|---|---|---|---|---|---|---|---|---|---|
| Aa2ADC | 100.00 | 97.86 | 89.11 | 88.75 | 82.52 | 82.52 | 84.74 | 86.58 | 85.13 |
| Aa3ADC | 97.86 | 100.00 | 88.93 | 88.57 | 81.98 | 82.16 | 84.92 | 86.23 | 84.41 |
| CtADC | 89.11 | 88.93 | 100.00 | 97.33 | 81.12 | 81.12 | 82.59 | 84.11 | 83.36 |
| CqADC | 88.75 | 88.57 | 97.33 | 100.00 | 80.94 | 80.94 | 82.29 | 83.99 | 83.54 |
| Aa4ADC | 82.52 | 81.98 | 81.12 | 80.94 | 100.00 | 98.05 | 89.07 | 89.17 | 89.70 |
| AdADC | 82.52 | 82.16 | 81.12 | 80.94 | 98.05 | 100.00 | 89.43 | 88.63 | 89.88 |
| AaADC | 84.74 | 84.92 | 82.59 | 82.29 | 89.07 | 89.43 | 100.00 | 92.06 | 92.72 |
| AsADC | 86.58 | 86.23 | 84.11 | 83.99 | 89.17 | 88.63 | 92.06 | 100.00 | 92.59 |
| As2ADC | 85.13 | 84.41 | 83.36 | 83.54 | 89.70 | 89.88 | 92.72 | 92.59 | 100.00 |

### Example 5: N-Terminal Truncations of Mosquito ADCs Resulted in Higher Beta-Alanine Production

Interestingly, the alignment in FIG. 2 revealed a region of low sequence conservation towards the amino terminus of the mosquito ADCs indicated in a broken line in FIG. 2, which immediately followed a 15-amino acid segment (SGSDSAGVSEDEDVQ; SEQ ID NO: 28) that was 100% conserved across all mosquito ADCs analyzed. To study the role of the N-terminus of CtADC in its activity, progressive N-terminal truncations were generated and expressed in bacteria, and their ADC activities were characterized as described in Example 1. Strikingly, N-terminal truncations ranging from 11 to 71 amino acids increased beta-alanine production by 24% to 100% as shown in Table 4. However, no ADC enzymatic activity was detected by truncating 81 amino acids or more from the N terminus of CtADC.

TABLE 4
Activities of N-terminal truncations of CtADC

| | AA sequence relative | |
|---|---|---|
| CtADC Truncation | to SEQ ID NO: 2 | Activity |
| CtADC (full length) | 1-561 | 2.5 |
| CtADCN1 | 12-561 | 3.55 |
| CtADCN2 | 22-561 | 4.2 |
| CtADCN3 | 32-561 | 4.95 |
| CtADCN4 | 42-561 | 4.8 |
| CtADCN5 | 52-561 | 3.4 |
| CtADCN6 | 62-561 | 4.8 |
| CtADCN7 | 72-561 | 3.1 |
| CtADCN8 | 82-561 | / |
| CtADCN9 | 92-561 | / |
| CtADCN10 | 102-561 | / |
| CtADCN11 | 131-561 | / |
| CtADCN12 | 171-561 | / |
| CtADCN13 | 201-561 | / |

“/”: Activity was too low to detect.

N-terminal truncations were also generated and characterized for another mosquito enzyme, AaADC as shown in Table 5. A 70% increase in beta-alanine production was observed by truncating the N-terminal 63 amino acids of AaADC. However, no ADC enzymatic activity was detected by truncating 137 amino acids or more of AaADC.

TABLE 5
Activities of N-terminal truncations of AaADCs

| | AA sequence relative | |
|---|---|---|
| AaADC Truncation | to SEQ ID NO: 4 | Activity |
| AaADC (full-length) | 1-561 | 2.0 |
| AaADCN1 | 64-568 | 3.4 |
| AaADCN2 | 138-568 | / |
| AaADCN3 | 208-568 | / |

“/”: Activity was too low to detect.

### Example 6: N-Terminal Truncations of Beetle ADCs Resulted in Higher Beta-Alanine Production

Progressive N-terminal truncations were generated and expressed in bacteria for two beetle ADCs and their ADC activities were characterized as described in Example 1. Results are shown in Tables 5 and 6 for AtADC and TcADC. For AtADC, a striking 256% increase in beta-alanine production was observed by truncating the N-terminal 45 amino acids. However, no ADC enzymatic activity was detected by truncating 114 amino acids or more of AtADC (Table 5). For TcADC, N-terminal truncations ranging from 10 to 50 amino acids increased beta-alanine production by 10% to 330%. However, no ADC enzymatic activity was detected by truncating 60 amino acids from the N terminus of TcADC (TcADCN6, Table 6).

TABLE 5
Activities of truncated AtADCs

| | AA sequence relative | |
|---|---|---|
| AtADC Truncation | to SEQ ID NO: 3 | Activity |
| AtADC (full length) | 1-544 | 1.6 |
| AtADCN1 | 46-544 | 4.1 |
| AtADCN2 | 115-544 | / |
| AtADCN3 | 185-544 | / |

“/”: Activity was too low to detect.

TABLE 6
Activities of truncated TcADCs

| | AA sequence relative | |
|---|---|---|
| TcADC Truncation | to SEQ ID NO: 1 | Activity |
| TcADC (full-length) | 1-540 | 1.0 |
| TcADCN1 | 11-540 | 2.8 |
| TcADCN2 | 21-540 | 1.1 |
| TcADCN3 | 31-540 | 1.25 |
| TcADCN4 | 41-540 | 3.3 |
| TcADCN5 | 51-540 | 2.3 |
| TcADCN6 | 61-540 | / |

“/”: Activity was too low to detect.

### Example 7: Analysis of Positions of N-Terminal Truncations Resulting in Higher Beta-Alanine Production

An alignment of the N-terminal sequences of the mosquito and beetle ADCs described in Examples 5 and 6 is shown in FIG. 3. The alignment in FIG. 3 helps visualize and understand the N-terminal truncation results in Examples 5 and 6, in which an N-terminal truncation between the two residues highlighted in black resulted in a truncated ADC having increased activity as compared to its corresponding full-length protein. Conversely, an N-terminal truncation between the two residues boxed in white resulted in a truncated ADC having no detectable ADC activity. Thus, with the truncation experiments for CtADC and TcADC providing the highest resolution, the area indicated with a broken line delineates the position where an N-terminal truncation is expected to cease being beneficial for beta-alanine production. The respective positions of the residues in the mosquito and beetle ADCs are shown in Table 7. The area marked with a broken line in FIG. 3 also coincides with the start of greater sequence conservation across the mosquito and beetle ADCs, with a tripeptide sequence “SLP” being 100% conversed across all the sequences aligned. Without wishing to be bound by theory, truncations N-terminal to (or upstream of) the serine within the conserved “SLP” tripeptide may be beneficial for increased beta-alanine production, while truncations downstream of the conserved serine may be detrimental (Table 7).

TABLE 7
Residue positions indicated in FIG. 3

| | | Position of area | Position of start |
|---|---|---|---|
| | SEQ ID | marked in a broken | of conserved |
| ADC | NO: | line in FIG. 3 | SLP tripeptide |
| CtADC | 2 | R72-E80 | S75 |
| AaADC | 4 | R79-E87 | S82 |
| AtADC | 3 | T56-A64 | S59 |
| TcADC | 1 | S52-R60 | S55 |
| Aa2ADC | 10 | R71-E79 | S74 |
| Aa3ADC | 11 | R71-E79 | S74 |
| CqADC | 9 | R74-E82 | S77 |
| Aa4ADC | 13 | R74-E82 | S77 |
| AdADC | 14 | R74-E82 | S77 |
| AsADC | 12 | R83-E91 | S86 |
| As2ADC | 15 | R79-E87 | S82 |
| TmADC | 6 | S53-E61 | S56 |
| AvADC | 5 | S57-E65 | S60 |

The N-terminal truncated CtADC sequence beginning at S75 was used as the basis for a further Protein BLAST™ search to identify other ADCs from various species. The ADC ortholog sequences sharing at least 70% amino acid identity with the truncated CtADC sequence were subjected to multiple sequence alignment analyses and a consensus ADC sequence is shown in SEQ ID NO: 29.

### Example 8: Comparison of CtADC with ADCs from Other Mosquito Species

CtADC outperformed other insect-derived ADCs by a significant margin from the enzyme activity testing performed in Example 2. Based on the activities shown in Table 2, CtADC exhibited a 25% increase in beta-alanine production over the next best insect-derived ADCs from mosquito (AaADCs) and fly (DmADC). Interestingly, CtADC shares about 97% overall amino acid sequence identity with CqADC (which is also derived from mosquito) yet the results in Table 2 reveal that CtADC exhibited 39% higher beta-alanine production than CqADC. The results shown in Table 4 reveal that at least the N-terminal 71 residues of CtADC may be truncated without abrogating ADC activity (CtADCN7). Thus, looking at amino acid differences between CtADC and CqADC within residues 72-561 of CtADC revealed only seven amino acid substitutions. Six of the seven amino acid substitutions correspond to residues that are found in different mosquito ADC orthologs. Interestingly, the only residue that was unique to CtADC was a glycine at position 96 (see residue highlighted in black in FIG. 2). In fact, a glycine corresponding to position 96 of the full-length CtADC (SEQ ID NO: 2) was not found in any other mosquito or beetle sequence analyzed (see FIG. 3), suggesting that this residue may play a role in the heightened beta-alanine production associated with CtADC

1. A recombinant truncated insect aspartate 1-decarboxylase (ADC), the truncated insect ADC lacking a sufficient number of contiguous residues within the amino terminal region of a corresponding full length wild-type insect ADC such that the truncated ADC exhibits increased conversion of aspartate to beta-alanine as compared to the corresponding full length wild-type insect ADC.

2. The recombinant truncated insect ADC of claim 1, which is a truncated variant of a mosquito, fly, beetle, flea, roach, or termite ADC; or which is a truncated variant of a wild-type ADC enzyme, the wild-type ADC enzyme being defined by an amino acid sequence at least 90, 91, 92, 93, 94, 95, 96, 97, 98, or 99% identical overall to the amino acid sequence of SEQ ID NO: 29.

3. The recombinant truncated insect ADC of claim 1, which is a truncated variant of an insect ADC from the genus: Culex, Anopheles, Drosophila, Aethina, Aedes, Tribolium, Anopheles, Tenebrio, Asbolus, or Cryptotermes.

4. The recombinant truncated insect ADC of claim 1, which is a truncated variant of an insect ADC from the species: Culex tarsalis, Anopheles arabiensis, Drosophila melanogaster, Culex quinquefasciatus, Aethina tumida, Aedes albopictus, Aedes aegypti, Tribolium castaneum, Anopheles sinensis, Tenebrio molitor, Asbolus verrucosus, or Cryptotermes secundus.

5. The recombinant truncated insect ADC of claim 1, wherein the corresponding full length wild-type insect ADC is:
(a) a mosquito ADC comprising an amino acid sequence at least 80, 81, 82, 83, 84, 85, 86, 87, 88, 89, 90, 91, 92, 93, 94, 95, 96, 97, 98, or 99% identical overall to any one of SEQ ID NOs: 2, 4, or 9-15;
(b) a beetle ADC comprising an amino acid sequence at least 80, 81, 82, 83, 84, 85, 86, 87, 88, 89, 90, 91, 92, 93, 94, 95, 96, 97, 98, or 99% identical overall to any one of SEQ ID NOs: 1, 3, or 5-6;
(c) a fly ADC comprising an amino acid sequence at least 80, 81, 82, 83, 84, 85, 86, 87, 88, 89, 90, 91, 92, 93, 94, 95, 96, 97, 98, or 99% identical overall to SEQ ID NO: 8; or
(d) an ADC comprising an amino acid sequence at least 90, 91, 92, 93, 94, 95, 96, 97, 98, or 99% identical overall to SEQ ID NO: 29.

6. The recombinant truncated insect ADC of claim 1, wherein the truncated ADC comprises an amino acid sequence at least 80, 81, 82, 83, 84, 85, 86, 87, 88, 89, 90, 91, 92, 93, 94, 95, 96, 97, 98, or 99% identical overall to:
(a) position 72 to 561 of the amino acid sequence of CtADC set forth in SEQ ID NO: 2;
(b) position 79 to 568 of the amino acid sequence of AaADC set forth in SEQ ID NO: 4;
(c) position 56 to 544 of the amino acid sequence of AtADC set forth in SEQ ID NO: 3;
(d) position 52 to 540 of the amino acid sequence of TcADC set forth in SEQ ID NO: 1;
(e) position 71 to 560 of the amino acid sequence of Aa2ADC set forth in SEQ ID NO: 10;
(f) position 71 to 562 of the amino acid sequence of Aa3ADC set forth in SEQ ID NO: 11;
(g) position 74 to 563 of the amino acid sequence of CqADC set forth in SEQ ID NO: 9;
(h) position 72 to 561 of the amino acid sequence of Aa4ADC set forth in SEQ ID NO: 13;
(i) position 74 to 624 of the amino acid sequence of AdADC set forth in SEQ ID NO: 14;
(j) position 83 to 572 of the amino acid sequence of AsADC set forth in SEQ ID NO: 12;
(k) position 72 to 561 of the amino acid sequence of As2ADC set forth in SEQ ID NO: 15;
(1) position 53 to 541 of the amino acid sequence of TmADC set forth in SEQ ID NO: 6;
(m) position 57 to 572 of the amino acid sequence of AvADC set forth in SEQ ID NO: 5; or
(n) position 1 to 491 the amino acid sequence set forth in SEQ ID NO: 29.

7. The recombinant truncated insect ADC of claim 1, wherein the truncated ADC comprises a glycine residue at a position corresponding to position 96 of the amino acid sequence of CtADC set forth in SEQ ID NO: 2.

8. The recombinant truncated insect ADC of claim 1, wherein the truncated ADC lacks at least X contiguous residues of the amino terminus of the corresponding full length wild-type insect ADC, wherein X is any integer between 5 and 50.

9. The recombinant truncated insect ADC of claim 1, wherein the truncation occurs at a position immediately C-terminal (downstream) of a residue corresponding to position n of a full-length wild-type insect ADC, wherein n is any integer between 2 and Y, wherein Y is the most C-terminal residue position within the full-length wild-type insect ADC at which truncation from the N terminus can occur with the truncated ADC exhibiting increased conversion of aspartate to beta-alanine as compared to the full length wild-type ADC.

10. The recombinant truncated insect ADC of claim 1, wherein the truncation occurs at a position C-terminal (downstream) of a residue corresponding to any one of:
(a) position 2, 3, 4, 5, 6, 7, 8, 9 10, or 11 to 71 of the amino acid sequence of CtADC set forth in SEQ ID NO: 2;
(b) position 2, 3, 4, 5, 6, 7, 8, 9 10, or 11 to 78 of the amino acid sequence of AaADC set forth in SEQ ID NO: 4;
(c) position 2, 3, 4, 5, 6, 7, 8, 9 10, or 11 to 55 of the amino acid sequence of AtADC set forth in SEQ ID NO: 3;
(d) position 2, 3, 4, 5, 6, 7, 8, 9 10, or 11 to 51 of the amino acid sequence of TcADC set forth in SEQ ID NO: 1;
(e) position 2, 3, 4, 5, 6, 7, 8, 9 10, or 11 to 70 of the amino acid sequence of Aa2ADC set forth in SEQ ID NO: 10;
(f) position 2, 3, 4, 5, 6, 7, 8, 9 10, or 11 to 70 of the amino acid sequence of Aa3ADC set forth in SEQ ID NO: 11;
(g) position 2, 3, 4, 5, 6, 7, 8, 9 10, or 11 to 73 of the amino acid sequence of CqADC set forth in SEQ ID NO: 9;
(h) position 2, 3, 4, 5, 6, 7, 8, 9 10, or 11 to 73 of the amino acid sequence of Aa4ADC set forth in SEQ ID NO: 13;
(i) position 2, 3, 4, 5, 6, 7, 8, 9 10, or 11 to 73 of the amino acid sequence of AdADC set forth in SEQ ID NO: 14;
(j) position 2, 3, 4, 5, 6, 7, 8, 9 10, or 11 to 82 of the amino acid sequence of AsADC set forth in SEQ ID NO: 12;
(k) position 2, 3, 4, 5, 6, 7, 8, 9 10, or 11 to 78 of the amino acid sequence of As2ADC set forth in SEQ ID NO: 15;
(1) position 2, 3, 4, 5, 6, 7, 8, 9 10, or 11 to 52 of the amino acid sequence of TmADC set forth in SEQ ID NO: 6;
(m) position 2, 3, 4, 5, 6, 7, 8, 9 10, or 11 to 56 of the amino acid sequence of AvADC set forth in SEQ ID NO: 5; or
(n) position 1 to 491 the amino acid sequence set forth in SEQ ID NO: 29.

11. The recombinant truncated insect ADC of claim 1, wherein the truncation occurs at a position N-terminal (upstream) of a residue corresponding to any one of:
(a) position 72 to 80 of the amino acid sequence of CtADC set forth in SEQ ID NO: 2;
(b) position 79 to 87 of the amino acid sequence of AaADC set forth in SEQ ID NO: 4;
(c) position 56 to 64 of the amino acid sequence of AtADC set forth in SEQ ID NO: 3;
(d) position 52 to 60 of the amino acid sequence of TcADC set forth in SEQ ID NO: 1;
(e) position 71 to 79 of the amino acid sequence of Aa2ADC set forth in SEQ ID NO: 10;
(f) position 71 to 79 of the amino acid sequence of Aa3ADC set forth in SEQ ID NO: 11;
(g) position 74 to 82 of the amino acid sequence of CqADC set forth in SEQ ID NO: 9;
(h) position 72 to 82 of the amino acid sequence of Aa4ADC set forth in SEQ ID NO: 13;
(i) position 74 to 82 of the amino acid sequence of AdADC set forth in SEQ ID NO: 14;
(j) position 83 to 91 of the amino acid sequence of AsADC set forth in SEQ ID NO: 12;
(k) position 72 to 87 of the amino acid sequence of As2ADC set forth in SEQ ID NO: 15;
(l) position 53 to 61 of the amino acid sequence of TmADC set forth in SEQ ID NO: 6;
(m) position 57 to 65 of the amino acid sequence of AvADC set forth in SEQ ID NO: 5; or
(n) position 1 to 491 the amino acid sequence set forth in SEQ ID NO: 29.

12. The recombinant truncated insect ADC of claim 1, wherein the truncation occurs at a position N-terminal (upstream) of a residue corresponding to any one of:
(a) position 75 of the amino acid sequence of CtADC set forth in SEQ ID NO: 2;
(b) position 82 of the amino acid sequence of AaADC set forth in SEQ ID NO: 4;
(c) position 59 of the amino acid sequence of AtADC set forth in SEQ ID NO: 3;
(d) position 55 of the amino acid sequence of TcADC set forth in SEQ ID NO: 1;
(e) position 74 of the amino acid sequence of Aa2ADC set forth in SEQ ID NO: 10;
(f) position 74 of the amino acid sequence of Aa3ADC set forth in SEQ ID NO: 11;
(g) position 77 of the amino acid sequence of CqADC set forth in SEQ ID NO: 9;
(h) position 77 of the amino acid sequence of Aa4ADC set forth in SEQ ID NO: 13;
(i) position 77 of the amino acid sequence of AdADC set forth in SEQ ID NO: 14;
(j) position 86 of the amino acid sequence of AsADC set forth in SEQ ID NO: 12;
(k) position 82 of the amino acid sequence of As2ADC set forth in SEQ ID NO: 15;
(l) position 56 of the amino acid sequence of TmADC set forth in SEQ ID NO: 6;
(m) position 60 of the amino acid sequence of AvADC set forth in SEQ ID NO: 5;
(n) position 1 to 491 the amino acid sequence set forth in SEQ ID NO: 29.

13. A recombinant protein having aspartate 1-decarboxylase activity, the recombinant protein comprising an amino acid sequence at least 80, 81, 82, 83, 84, 85, 86, 87, 88, 89, 90, 91, 92, 93, 94, 95, 96, 97, 98, or 99% identical overall to:
(a) position 72 to 561 of the amino acid sequence of CtADC set forth in SEQ ID NO: 2;
(b) position 79 to 568 of the amino acid sequence of AaADC set forth in SEQ ID NO: 4;
(c) position 56 to 544 of the amino acid sequence of AtADC set forth in SEQ ID NO: 3;
(d) position 52 to 540 of the amino acid sequence of TcADC set forth in SEQ ID NO: 1;
(e) position 71 to 560 of the amino acid sequence of Aa2ADC set forth in SEQ ID NO: 10;
(f) position 71 to 562 of the amino acid sequence of Aa3ADC set forth in SEQ ID NO: 11;
(g) position 74 to 563 of the amino acid sequence of CqADC set forth in SEQ ID NO: 9;
(h) position 72 to 561 of the amino acid sequence of Aa4ADC set forth in SEQ ID NO: 13;
(i) position 74 to 624 of the amino acid sequence of AdADC set forth in SEQ ID NO: 14;
(j) position 83 to 572 of the amino acid sequence of AsADC set forth in SEQ ID NO: 12;
(k) position 72 to 561 of the amino acid sequence of As2ADC set forth in SEQ ID NO: 15;
(l) position 53 to 541 of the amino acid sequence of TmADC set forth in SEQ ID NO: 6;
(m) position 57 to 572 of the amino acid sequence of AvADC set forth in SEQ ID NO: 5; or
(n) position 1 to 491 the amino acid sequence set forth in SEQ ID NO: 29.

14. The recombinant protein of claim 13, which comprises a glycine residue at a position corresponding to position 96 of the amino acid sequence of CtADC set forth in SEQ ID NO: 2.

15. A polynucleotide comprising a nucleic acid sequence encoding the recombinant truncated insect ADC as defined in claim 1.

16. An expression cassette comprising the isolated or recombinant polynucleotide of claim 15 operably linked to a promoter that is heterologous with respect to the insect ADC.

17. A host cell that expresses the recombinant truncated insect ADC as defined in claim 1.

18. The host cell of claim 17, which is a bacterial, insect, mammalian, yeast or fungal cell.

19. (canceled)

20. A process for the production of beta-alanine, the process comprising:
(a) providing an ADC enzyme source which is the truncated insect ADC as defined in claim 1;
(b) contacting the ADC enzyme source with a source of aspartate under conditions enabling the ADC enzyme source to catalyze the conversion of the aspartate to beta-alanine; and
(c) isolating and/or concentrating the beta-alanine produced.

21. The process of claim 20, wherein the ADC enzyme source is an intact host cell.

22. (canceled)

23. (canceled)


SEQUENCE LISTING (29 sequences)

SEQ ID NO: 1 - protein, 540 aa, Tribolium castaneum
MPATGEDQDLVQDLIEEPATFSDAVLSSDEELFHQKCPKPAPIYSPVSKPVSFESLPNRRLHEEFLRSSVDVLLQEAVFEGTNRKNRVLQWREPEELRRLMDFGVRSAPSTHEELLEVLKKVVTYSVKTGHPYFVNQLFSAVDPYGLVAQWATDALNPSVYTYEVSPVFVLMEEVVLREMRAIVGFEGGKGDGIFCPGGSIANGYAISCARYRFMPDIKKKGLHSLPRLVLFTSEDAHYSIKKLASFQGIGTDNVYLIRTDARGRMDVSHLVEEIERSLREGAAPFMVSATAGTTVIGAFDPIEKIADVCQKYKLWLHVDAAWGGGALVSAKHRHLLKGIERADSVTWNPHKLLTAPQQCSTLLLRHEGVLAEAHSTNAAYLFQKDKFYDTKYDTGDKHIQCGRRADVLKFWFMWKAKGTSGLEKHVDKVFENARFFTDCIKNREGFEMVIAEPEYTNICFWYVPKSLRGRKDEADYKDKLHKVAPRIKERMMKEGSMMVTYQAQKGHPNFFRIVFQNSGLDKADMVHLVEEIERLGSDL

SEQ ID NO: 2 - protein, 561 aa, Culex tarsalis
MPTNGMLDVALQVIEDANLSSGSDSAGVSEDEDVQLFSTTGNIVSSKPLKKPALKPATKDEDQNKTKANAKRYASLPNREQHQRFLTDFLSEVLNGAIFNATDRSNKVLNWVDPEELKRSIDLSLKDEPDSDEKLLELARATIDHSVKTGHPYFMNQLFSSVDPYGFAGQVLTDALNPSVYTFEVSPVFVLMEEVVLKEMRTIVGFPGGVGDGIFCPGGSMANGYAISCARFKHMPDVKTKGLHSLPRLVIFTSEDAHYSIKKLASFMGIGSDNVYPIRTDAVGKIQPDHLEAEILRAKSEGAVPFMVSATAGTTVIGAFDPLEQIADLCQKYNLWMHVDAAWGGGALMSKKYRTLLKGVERADSVTWNPHKLLAAPQQCSTFLTRHEGILSGCHSTNATYLFQKDKFYDTQYDTGDKHIQCGRRADVLKFWFMWRAKGTSGLEQHIDKVFETAEYFTNSIKARPGFEMVIENPECTNVCFWYVPPGLRQVPRDSAEFGERLHKVAPKVKERMMREGSMMITYQPIHDKPNFFRLVLQNSALDKSDMNYIIDEIERLASDL

SEQ ID NO: 3 - protein, 544 aa, Aethina tumida
MPANGQLEDGFHLIDEPATYSDAVASSSDDETVQYSNDERSIRDMKATIATGKLATFESLPSRAHHEEFIRSCMDVILKEAVFDGTNRNNPVLNFVNPEELQSKVNFKLKTAPSTHEDLLKTLKDTIRYSVKTGHPYFVNQLFSSLDPYGLVGQWLTDALNPTVYTYEVSPVFTLMEEEVLREMRTIVGFKNGEGDGIFCPGGSMANGYAISCARHKFIPDIKKKGLHALPRLVLFTSQDAHYSIKKLSSFLGLGTDNVYAICTDAKGKMDVGHLVEEIERALEEGAAPFMVSATSGTTVIGAFDPLDEIADVCQKYGLWMHVDAAWGGGALMSKKHRHLLKGVERADSVTWNPHKLLTAPQQCSTLLLRHEGLLAECNSANATYLFQKDKFYDTKYDLGDKHIQCGRRPDVLKFWFMWKAKGTSGFEQHIDKVFENTKYFTDSIKNRPGFELVVPEPECTNICFWYVPPSLRQAKSDPDYKEKLHKVAPKIKERMMKEGSMMVTYQPLREVPNFFRIVFQNSGLNKTDMTHLIEEFERLGHDL

SEQ ID NO: 4 - protein, 568 aa, Anopheles arabiensis
MPANGVCSVGLEVIEDNATYASGSDSAGVSEDEDVQQLFVSGADRVTSVLPKKSDIRKASQVDEQAAAAAAAAAVSEKRYASLPNREQHQQFLTQFLTEVLNSAVFNATDRANKVLNWVDPEELQRTLDLALKDEPDTHEKLLELTRATIRHSVKTGHPYFMNQLFSSVDPYGFAGQVLTDALNPSVYTYEVSPVFVLMEEVVLREMRTIVGYPDGEGDGIFAPGGSMANGYAISCARHKFMPDIKTKGLHALPRLVIFTSEDAHYSVKKLASFMGIGSDNVYAIKTDNVGKIRVEHLESEILRAKSEGALPFMVSATAGTTVIGAFDPLEQIADLCAKYNLWMHVDAAWGGGALMSKKYRTLLKGIERSDSVTWNPHKLLAAPQQCSTLLTRHRNILAEAHSTNATYLFQKDKFYDTRYDTGDKHIQCGRRADVLKFWFMWRAKGTAGFEAHIDKVFENAEHFTSSIKARPGFEMVIEQPECTNVCFWYVPPGLRGVPRDSAEYRDRLHKVAPKVKERMMKDGSMMITYQPIHDKPNFFRLVLQNSSLDKSDMNYIIDEIERLGKDL

SEQ ID NO: 5 - protein, 572 aa, Asbolus verrucosus
MPATGEQDDLVQDIIEEPATYSDAVLSSDDEVCVRYSSQSDTNNSSFYQTATKKLASFESLPNREHHEDFIKKCAEILIREAVFEGTNRKNRVLQWNSPEELQKLMDFTLRTSPSSHDELLDLLRNTVNYSVKTGHPYFVNQLFSSLDPYGLVGQWATDALNPSVYTYEVSPVFTLMEEVVLREMRTIVGFEGGRGDGIFCPGGSIANGYAISCARHKFQPEIKATASVTNLLKNIANIILLLLQTKGLHSLPRLVLFTSEDAHYSIKKLSSFLGIGTDNVYLIRTDDRGRMDPSHLIQEIERALAEGGAPFMVSATAGTTVIGAFDPIDQIADICEKYNLWLHVDAAWGGGALMSSKHRSLLKGIERADSVTWNPHKLLTAPQQCSTLLLRHEGLLSETHSTHAAYLFQKDKFYDTKFDTGTKKFNGDKHIQCGRRADVLKFWFMWKAKGTLGFEKHINKVFDNAKFFADSIRNRVGFEMLIDQPECTNVCFWYIPESLRNAKQDSDYKERLHKVAPKIKERMMKEGSMMVTYQAQKSHPNFFRIVFQSSGLDRADMLHLIEEFERLGRDL

SEQ ID NO: 6 - protein, 541 aa, Tenebrio molitor
MPARGEQDDVVQDIIEEPATYGDAILSSDDEVYTKFSERPLTQFYQPSQKRASFESLPNRERHEEFIRKSVEILLKDAVFEGTSRNNRVLQWTCPEELSRLMEFGLKNGPSTHEELLEILKKVVNYSVKTGHPYFVNQLFSSLDPYGLVAQWATDALNPSVYTYEVSPVFILMEEVVLKEMRSIVGFEAGRGDGIFCPGGSIANGYAISCARYKFQPDIKRKGLHSLPRLVLFTSEDAHYSIKKLSSFLGIGTDNVYLIRTDDRGRMDVTHLIGQIERSLSEGAAPFMVSATAGTTVIGAFDPLNEIASVCEKYKLWLHVDAAWGGGALVSGKHKSLLKGIERADSVTWNPHKLLTAPQQCSTLLLRHEGILAAAHSTNAAYLFQKDKSYDTKFDTGDKHIQCGRRADVLKFWFMWKAKGTSGLEKHINKVFENAAYFADSIRNREGFEMVIDQPECTNVCFWYIPESLRSCKQDSDYKERLHKVAPKIKERMMKEGSMMVTYQAQKQHPNFFRIVFQNSGLDKADMIHFVEEIERLGKDL

SEQ ID NO: 7 - protein, 547 aa, Cryptotermes secundus
MPASSGIITLTQSLENLNGKHGISGSYEDMTAGVNVAVPSLSPSPGYVTEKKSTRSVAWFASLPDRQRHSQFLKEAVDLMLDKAVFDAASRTNRVVEWRSPEELKKLIDLDLPADRVSHDRLLQLLKDIIQYSVKTGHPYFVNQLFSSVDPYGLVGQWLGDALNPSVYTYEVSPVFTLMEETVLCEMRRIVGFPEGRGDGIFCPGGSIANGYAISCARYNFVPDVKKRGLHGLPRLVLFTSEDAHYSIKKMASLLGLGSDNVYLIHCNSKGKMDVQHLEQEIQRALEEGAAPFMVSATAGTTVLGAFDPIPKIADICSKYKMWLHVDAAWGGGALVSKKHKHLLEGIEKADSVTWNPHKLLTAPQQCSTFLLRHEGVLSACHSASAQYLFQKDKFYDTQYDTGDKHIQCGRRADVLKFWFMWKAKGTVGLEEHIDTVFDNAAYFTKQIKKREGFRMVLQEPECTNVCFWYIPPSLRGHEDQSDFSERLHKVAPRIKERMIKEGSMMVTYQPLRDQPNFFRLVLQNSGLDWADMDYFVQEFERLGSDL

SEQ ID NO: 8 - protein, 575 aa, Drosophila melanogaster
MLASENFPTHHFKESIFKPYSTTSGDDLASVSPLTATAALVASTSSPADSTSTVAFEQASKMLANAANNNNNNNNNITSTKDDLSSFVASHPAAEFEGFIRACVDEIIKLAVFQGTNRSSKVVEWHEPAELRQLFDFQLREQGESQDKLRELLRETIRFSVKTGHPYFINQLYSGVDPYALVGQWLTDALNPSVYTYEVAPLFTLMEEQVLAEMRRIVGFPNGGQGDGIFCPGGSIANGYAISCARYRHSPESKKNGLFNAKPLIIFTSEDAHYSVEKLAMFMGFGSDHVRKIATNEVGKMRLSDLEKQVKLCLENGWQPLMVSATAGTTVLGAFDDLAGISEVCKKYNMWMHVDAAWGGGALMSKKYRHLLNGIERADSVTWNPHKLLAASQQCSTFLTRHQQVLAQCHSTNATYLFQKDKFYDTSFDTGDKHIQCGRRADVFKFWFMWKAKGTQGLEAHVEKVFRMAEFFTAKVRERPGFELVLESPECTNISFWYVPPGLREMERNREFYDRLHKVAPKVKEGMIKKGSMMITYQPLRQLPNFFRLVLQNSCLEESDMVYFLDEIESLAQNL

SEQ ID NO: 9 - protein, 563 aa, Culex quinquefasciatus
MPTNGMFDVALQVIEDANLSSGSDSAGVSEDEDVQLFCTTGNVVSSKPLKKPSLKPVTTVKDEDQNKMKTNAKRYASLPNREQHQRFLTDFLSEVLNNAIFNATDRSNKVLNWVDPEELKRSIDLSLKAEPDSDEKLLELARATIDHSVKTGHPYFMNQLFSSVDVYGFAGQCLTDALNPSVYTFEVSPVFVLMEEVVLKEMRTIVGFPGGVGDGIFCPGGSMANGYAISCARFKHMPDVKTKGLHSLPRLVIFTSEDAHYSIKKLASFMGIGSDNVYPIRTDAVGKIQPDHLEAEILRAKSEGALPFMVSATAGTTVIGAFDPLEQIADLCQKYNLWMHVDAAWGGGALMSKKYRTLLKGVERADSVTWNPHKLLAAPQQCSTFLTRHEGILSGCHSTNATYLFQKDKFYDTQYDTGDKHIQCGRRADVLKFWFMWRAKGTSGFEQHIDKVFENAEYFTNSIKARPGFEMVIENPECTNVCFWYVPPGLRQVPRDSAEFGERLHKVAPKVKERMMREGSMMITYQPIHDKPNFFRLVLQNSGLDKSDMNYIIDEIERLASDL

SEQ ID NO: 10 - protein, 560 aa, Aedes albopictus
MPANGMFDVALQVIDDSNVSSGSDSAGVSEDEDVQLFCSMGNTIAPKPLKKSITKTKDEEFSKTAKANEKRYASLPNREQHQQFLTDFLSEVLNNAVFNATERANKVLNWVDPEQLKRTLDLELKDEPDSHEKLLELTRATIKHSVKTGHPYFMNQLFSSVDPYGFAGQILTDALNPSVYTFEVSPVFVLMEEVVLKEMRTIVGYPDGAGDGIFCPGGSMANGYSISCARFKHMPDVKTKGLHSLPRLVIFTSEDAHYSVKKLASFMGIGSDNVYPIRTDAIGKIRVDHLESEILRAKAEGAVPFMVSATAGTTVIGAFDPLEQIADLCKKYNLWMHVDAAWGGGALMSKKYRSLLKGIERSDSVTWNPHKLLAAPQQCSTFLTRHEGILSECHSTNATYLFQKDKFYDTQYDTGDKHIQCGRRADVLKFWFMWRAKGTSGLEQHIDKVFENAEHFTNSIKARDGFEMVVETPECTNVCFWYVPPGLRSVPRDSAEFTERLHKVAPKVKERMMREGSMMITYQPIHDKPNFFRLVLQNSALDKSDMNYIIDEIERLAADL

SEQ ID NO: 11 - protein, 562 aa, Aedes aegypti
MPANGMFDVALQVIDDSNVSSGSDSAGVSEDEDVQLFCSKGNTIVPKPLKKSISKIKDEEFSKTAKANEKRYASLPSREHHQQFLTDFLSEVLNNAVFNATERANKVLNWVDPEQLKRTLDLELKDEPDSHEKLLELTRATIKHSVKTGHPYFMNQLFSSVDPYGFAGQILTDALNPSVYTFEVSPVFVLMEEVVLKEMRTIVGYPDGTGDGIFCPGGSMANGYSISCARFKHMPDVKTKGLHSLPRLVIFTSEDAHYSVKKLASFMGIGSDNVYPIRTDAIGKIRVDHLESEILRAKSEGAVPFMVSATAGTTVIGAFDPLEQIADLCKKYNLWMHVDAAWGGGALMSKKYRSLLKGIERSDSVTWNPHKLLAAPQQCSTFLTRHEGILSECHSTNATYLFQKDKFYDTQYDTGDKHIQCGRRADVLKFWFMWRAKGTSGLEQHIDKVFENAEHFTSSIKAREGFEMVVENPECTNVCFWYVPPGLRNVPRDSAEFTERLHKVAPKVKERMMREGSMMITYQPIHDKPNFFRLVLQNSALDKSDMNYIIDEIERLAADLKP

SEQ ID NO: 12 - protein, 572 aa, Anopheles sinensis
MPANGVNSVELEVIEDVATTYASGSDSAGVSEDEDVQQLFVSGAHHISSVPPLKKAVETRGKGTQLQGPASEGAAAAEVSEKRYASLPNREQHQQFLTDFLTEVLNSAVFNATDRANKVLNWVDPEELKRTLDLAIKQEPDTHEKLLELTRATIRHSVKTGHPYFMNQLFSSVDPYGFAGQVLTDALNPSVYTFEVSPVFVLMEEVVLREMRTIVGYPNGEGDGIFAPGGSMANGYAISCARYKFMPDVKAKGLHALPRLVIFTSEDAHYSVKKLASFMGIGSDNVYAIKTDAIGKICVDHLESEILRAKQEGALPFMVSATAGTTVIGAFDPLEQIADLCAKYNLWMHVDAAWGGGALMSKKYRTLLKGIERSDSVTWNPHKLLAAPQQCSTLLTRHRNILSECHSTNATYLFQKDKFYDTRYDTGDKHIQCGRRADVLKFWFMWRAKGTAGFEQHIDKVFENAEHFTSSIKARPGFEMVIENPECTNVCFWYVPPGLRSVPRDSAEFRERLHKVAPKVKERMMKEGSMMITYQPIHDKPNFFRLVLQNSSLDKSDMNYIIDEIERLGKDL

SEQ ID NO: 13 - protein, 563 aa, Anopheles albimanus
MPATGVSSIGLEVQEEPATYASGSDSAGVSEDEDVQQLFVNGAHGLTSVAPAVRKPEMRGKLSLDESAAIDRKRYASLPNREQHQQFLTEFLTEVLNSAVFNATDRANKVLNWVDPEELSRTLDLAIKDEPDTHERLLELTRATIRHSVKTGHPYFMNQLFSSVDPYGFAGQVLTDALNPSVYTYEVSPVFVLMEETVLREMRKIVGYPNGVGDAIFAPGGSMANGYAISCARHKFMPDIKAKGLHALPRLVIFTSEDAHYSIKKLASFMGIGSDNVYPIKTDEIGKICVDHLESEILRAKAEGASPFMVSATAGTTVIGAFDPLEQIADLCEKYQLWFHVDAAWGGGALMSKKYRTLLKGIERSDSVTWNPHKLLAAPQQCSTLLTRHPNLLSECHSTNATYLFQKDKFYDTQYDTGDKHIQCGRRADVLKFWFMWRAKGSTGFEQHIDKVFENAEYFTRSIKARPGFEMVIEHPECTNVCFWYVPPSLRDMARDSAEYRERLHKVAPKVKERMMKEGSMMITYQPIHDKPNFFRLVLQNSSLDKSDMNYIIDEIERLGKDL

SEQ ID NO: 14 - protein, 624 aa, Anopheles darlingi
MPATGVSSIGLEVHEEPATYASGSDSAGVSEDEDVQQLFVNGAHGVTRVAPAARKAEMRGKLSLDESAAIDRKRYASLPNREQHQQFLTEFLTEVLNSAVFNATDRANKVLNWVDPEELSRTLDLAIKDEPDTHERLLELTRATIRHSVKTGHPYFMNQLFSSVDPYGFAGQVLTDALNPSVYTYEVSPVFVLMEETVLREMRKIVGYPNGVGDAIFAPGGSMANGYAISCARHKFMPDIKGKSFRTMHLITLIESAGYGMTIVSQHVTTVVAAIKIVHRQRRSTGCYTRSWLIETIGNQASAKGLHALPRLVIFTSEDAHYSIKKLASFMGIGSDNVYPIKTDDIGKIRVDHLESEILRARAEGALPFMVSATAGTTVIGAFDPLEQIADLCEKYQLWFHVDAAWGGGALMSKKYRTLLKGIERSDSVTWNPHKLLAAPQQCSTLLTRHPNLLSECHSTNATYLFQKDKFYDTQYDTGDKHIQCGRRADVLKFWFMWRAKGSTGFEQHIDKVFENAEYFTRSIKARPGFEMVIEHPECTNVCFWYVPPSLRGMARDSAEYRERLHKVAPKVKERMMKEGSMMITYQPIHDKPNFFRLVLQNSSLDKSDMNYIIDEIERLGKDL

SEQ ID NO: 15 - protein, 568 aa, Anopheles stephensi
MPANGVCSVGLEVIEDNAATYASGSDSAGVSEDEDVQQLFVNGADRVTSVSSLPKKSTEARGKLSQHGDDGKPAVAEKRYASLPNREQHQQFLTEFLTEVLNSAVFNATDRSNKVLNWVDPEELKRTLDLAIKDEPDTHEKLLELTRATIRHSVKTGHPYFMNQLFSSVDPYGFAGQVLTDALNPSVYTFEVSPVFVLMEEVVLREMRSIVGYPNGEGDGIFAPGGSMANGYAISCARHKFMPDIKTKGLHALPRLVIFTSEDAHYSVKKLASFMGIGSDNVYAIKTDSIGKIRIEHLESEILRAKAEGALPFMVSATAGTTVIGAFDPLEQIADLCAKHNLWMHVDAAWGGGALMSKKYRTLLKGIERSDSVTWNPHKLLAAPQQCSTLLTRHRNILSECHSTNATYLFQKDKFYDTRYDTGDKHIQCGRRADVLKFWFMWRAKGTAGFEQHIDKVFENAEHFTNSIKARPGFEMVIENPECTNVCFWYVPPGLRSVPRDSSEFRERLHKVAPKVKEHMMKEGSMMITYQPIHDKPNFFRLVLQNSSLDKSDMNYIIDEIERLGKDL

SEQ ID NO: 16 - DNA, 1623 bp, Tribolium castaneum
atgccggcgaccggcgaggaccaggatctggttcaagacctgatcgaggaaccggcgaccttcagcgatgcggtgctgagcagcgacgaggaactgtttcaccagaaatgcccgaagccggcgccgatttacagcccggtgagcaaaccggttagcttcgaaagcctgccgaaccgtcgtctgcacgaggaatttctgcgtagcagcgtggatgttctgctgcaggaagcggtgttcgaaggcaccaaccgtaagaaccgtgttctgcaatggcgtgagccggaggaactgcgtcgtctgatggactttggtgttcgtagcgcgccgagcacccatgaggaactgctggaagtgctgaagaaagtggttacctacagcgtgaaaaccggccacccgtatttcgttaaccagctgtttagcgcggtggatccgtacggtctggttgcgcagtgggcgaccgatgcgctgaacccgagcgtgtacacctatgaggttagcccggtgttcgttctgatggaggaagtggttctgcgtgagatgcgtgcgatcgttggtttcgaaggtggcaagggcgatggtatcttctgcccgggtggcagcattgcgaacggttacgcgattagctgcgcgcgttatcgttttatgccggacatcaagaaaaagggtctgcacagcctgccgcgtctggttctgttcaccagcgaagatgcgcactacagcattaaaaagctggcgagctttcagggcatcggtaccgacaacgtgtatctgattcgtaccgatgcgcgtggccgtatggacgtgagccacctggttgaggaaattgagcgtagcctgcgtgagggtgcggcgccgtttatggtgagcgcgaccgcgggtaccaccgttattggtgcgtttgatccgatcgagaaaattgcggacgtgtgccaaaaatacaagctgtggctgcatgttgatgcggcgtggggtggcggtgcgctggtgagcgcgaaacaccgtcacctgctgaagggcatcgaacgtgcggacagcgttacctggaacccgcacaagctgctgaccgcgccgcagcaatgcagcaccctgctgctgcgtcacgagggtgtgctggcggaagcgcacagcaccaacgcggcgtacctgttccagaaggataagttttacgacaccaaatatgacaccggcgataagcacatccaatgcggtcgtcgtgcggatgttctgaaattctggtttatgtggaaagcgaagggcaccagcggtctggagaaacacgtggataaggttttcgaaaacgcgcgtttctttaccgactgcattaagaaccgtgagggcttcgaaatggttatcgcggagccggaatacaccaacatttgcttttggtacgttccgaaaagcctgcgtggtcgtaaggacgaggcggattacaaagacaagctgcacaaagtggcgccgcgtatcaaagagcgtatgatgaaggaaggcagcatgatggttacctatcaggcgcaaaaaggtcacccgaacttctttcgtatcgtgttccagaacagcggcctggataaggcggacatggtgcatctggtggaggaaattgaacgtctgggtagcgacctgtaa

SEQ ID NO: 17 - DNA, 1686 bp, Culex tarsalis
atgccgaccaacggcatgctggacgtggcgctgcaagttattgaggatgcgaacctgagcagcggcagcgacagcgcgggtgtgagcgaggacgaagatgttcaactgttcagcaccaccggtaacatcgtgagcagcaaaccgctgaagaaaccggcgctgaagccggcgaccaaagacgaagatcagaacaagaccaaagcgaacgcgaagcgttacgcgagcctgccgaaccgtgagcagcaccaacgtttcctgaccgactttctgagcgaagttctgaacggcgcgatctttaacgcgaccgaccgtagcaacaaagtgctgaactgggttgatccggaggaactgaagcgtagcattgacctgagcctgaaagatgagccggacagcgatgagaagctgctggaactggcgcgtgcgaccatcgaccacagcgtgaagaccggtcacccgtacttcatgaaccagctgtttagcagcgtggacccgtatggcttcgcgggtcaagttctgaccgatgcgctgaacccgagcgtgtacaccttcgaagttagcccggtgtttgttctgatggaggaagtggttctgaaagaaatgcgtaccattgtgggtttcccgggtggcgttggcgacggtatcttttgcccgggtggcagcatggcgaacggctatgcgattagctgcgcgcgttttaagcacatgccggacgtgaagaccaaaggtctgcacagcctgccgcgtctggttattttcaccagcgaagatgcgcactacagcatcaagaaactggcgagctttatgggcatcggtagcgataacgtgtatccgattcgtaccgacgcggttggcaaaatccagccggatcacctggaggcggaaattctgcgtgcgaagagcgagggtgcggtgccgttcatggttagcgcgaccgcgggcaccaccgtgattggtgcgtttgacccgctggaacagatcgcggatctgtgccaaaaatacaacctgtggatgcatgttgatgcggcgtggggtggcggtgcgctgatgagcaagaaatatcgtaccctgctgaaaggtgtggagcgtgcggatagcgttacctggaacccgcacaagctgctggcggcgccgcagcaatgcagcaccttcctgacccgtcacgaaggcattctgagcggttgccacagcaccaacgcgacctacctgttccagaaggacaaattttacgatacccaatatgacaccggcgataagcacattcagtgcggtcgtcgtgcggacgttctgaaattctggtttatgtggcgtgcgaagggtaccagcggtctggagcaacacatcgataaagtgttcgagaccgcggaatactttaccaacagcattaaggcgcgtccgggcttcgaaatggttatcgagaacccggaatgcaccaacgtgtgcttttggtatgttccgccgggtctgcgtcaagtgccgcgtgacagcgcggagttcggtgaacgtctgcacaaagtggcgccgaaggttaaagagcgtatgatgcgtgaaggtagcatgatgatcacctaccagccgattcacgataaaccgaacttctttcgtctggttctgcaaaacagcgcgctggacaagagcgatatgaactatatcattgacgagatcgaacgtctggcgagcgatctgtaa

SEQ ID NO: 18 - DNA, 1635 bp, Aethina tumida
atgccggcgaacggtcagctggaagacggcttccacctgattgatgaaccggcgacctatagcgatgcggtggcgagcagcagcgatgatgaaaccgttcaatatagcaacgacgagcgtagcatccgtgatatgaaagcgaccattgcgaccggcaagctggcgaccttcgaaagcctgccgagccgtgcgcaccacgaggaatttatccgtagctgcatggacgtgattctgaaagaggcggttttcgatggcaccaaccgtaacaacccggtgctgaactttgttaacccggaggaactgcaaagcaaagtgaacttcaaactgaagaccgcgccgagcacccacgaagacctgctgaaaaccctgaaggataccattcgttacagcgtgaagaccggtcacccgtatttcgttaaccagctgtttagcagcctggacccgtacggtctggtgggccaatggctgaccgatgcgctgaacccgaccgtttacacctatgaggtgtctccggtttttaccctgatggaggaagaggtgctgcgtgaaatgcgtaccatcgttggcttcaagaacggtgaaggtgatggtatcttctgcccgggtggcagcatggcgaacggttatgcgatcagctgcgcgcgtcacaaattcatcccggatattaagaaaaagggcctgcatgcgctgccgcgtctggtgctgtttaccagccaggacgcgcactacagcatcaaaaagctgagcagcttcctgggtctgggcaccgataacgtttatgcgatttgcaccgacgcgaaaggcaagatggatgtgggccacctggttgaagagattgaacgtgcgctggaagagggtgcggcgccgtttatggttagcgcgaccagcggtaccaccgttatcggcgcgttcgacccgctggatgagattgcggacgtgtgccaaaaatacggtctgtggatgcatgttgatgcggcgtggggtggcggtgcgctgatgagcaaaaagcaccgtcacctgctgaagggcgtggaacgtgcggacagcgttacctggaacccgcacaaactgctgaccgcgccgcagcaatgcagcaccctgctgctgcgtcacgaaggtctgctggcggagtgcaacagcgcgaacgcgacctacctgttccagaaagacaagttttacgataccaaatatgacctgggtgataagcacatccaatgcggccgtcgtccggatgtgctgaagttctggtttatgtggaaagcgaagggtaccagcggcttcgaacagcacatcgacaaagttttcgagaacaccaagtattttaccgatagcattaaaaaccgtccgggttttgaactggtggttccggaaccggagtgcaccaacatttgcttctggtacgttccgccgagcctgcgtcaagcgaaaagcgacccggattataaagagaagctgcacaaggtggcgccgaaaatcaaggaacgtatgatgaaagagggcagcatgatggttacctaccagccgctgcgtgaagtgccgaacttctttcgtatcgtttttcaaaacagcggtctgaacaagaccgacatgacccacctgattgaagagttcgagcgtctgggccacgatctgtaa

SEQ ID NO: 19 - DNA, 1707 bp, Anopheles arabiensis
atgccggcgaacggtgtgtgcagcgttggcctggaagtgattgaagacaacgcgacctacgcgagcggtagcgatagcgcgggcgttagcgaggacgaagatgtgcagcaactgttcgttagcggtgcggaccgtgtgaccagcgttctgccgaagaaaagcgacatccgtaaagcgagccaggtggatgagcaagcggcggcggcggcggcggcggcggcggtgtctgagaagcgttatgcgagcctgccgaaccgtgaacagcaccagcaattcctgacccaatttctgaccgaagtgctgaacagcgcggtttttaacgcgaccgaccgtgcgaacaaagtgctgaactgggttgacccggaggaactgcaacgtaccctggatctggcgctgaaggacgagccggatacccacgagaaactgctggaactgacccgtgcgaccattcgtcacagcgtgaagaccggtcacccgtacttcatgaaccagctgtttagcagcgtggacccgtatggtttcgcgggccaagttctgaccgatgcgctgaacccgagcgtgtacacctatgaagttagcccggtgtttgttctgatggaggaagtggttctgcgtgagatgcgtaccattgttggctacccggacggcgaaggtgatggtatttttgcgccgggtggcagcatggcgaacggttatgcgattagctgcgcgcgtcacaaatttatgccggacatcaagaccaagggtctgcatgcgctgccgcgtctggtgattttcaccagcgaggatgcgcactacagcgttaagaaactggcgagctttatgggtatcggcagcgacaacgtgtatgcgattaagaccgataacgtgggtaaaatccgtgttgagcacctggagagcgaaatcctgcgtgcgaaaagcgaaggtgcgctgccgttcatggttagcgcgaccgcgggtaccaccgttattggcgcgtttgacccgctggaacagatcgcggatctgtgcgcgaagtacaacctgtggatgcatgtggatgcggcgtggggtggcggtgcgctgatgagcaagaaatatcgtaccctgctgaagggtattgagcgtagcgatagcgttacctggaacccgcacaaactgctggcggcgccgcagcaatgcagcaccctgctgacccgtcaccgtaacatcctggcggaagcgcacagcaccaacgcgacctacctgttccagaaggacaaattttacgatacccgttatgacaccggtgataagcacattcaatgcggccgtcgtgcggacgttctgaagttctggtttatgtggcgtgcgaaaggtaccgcgggcttcgaggcgcacatcgataaggtgttcgagaacgcggaacactttaccagcagcattaaagcgcgtccgggtttcgaaatggttatcgagcaaccggaatgcaccaacgtgtgcttttggtatgttccgccgggtctgcgtggcgtgccgcgtgacagcgcggagtatcgtgatcgtctgcacaaggtggcgccgaaggttaaagaacgtatgatgaaagacggtagcatgatgatcacctaccagccgattcacgataagccgaacttctttcgtctggttctgcaaaacagcagcctggacaaaagcgatatgaactatatcattgacgagatcgaacgtctgggcaaggatctgtaa

SEQ ID NO: 20 - DNA, 1738 bp, Asbolus verrucosus
tggccatatgccggcgaccggcgagcaggacgatctggtgcaagatatcattgaggaaccggcgacctacagcgacgcggttctgagcagcgacgatgaagtgtgcgttcgttacagcagccagagcgataccaacaacagcagcttttatcaaaccgcgaccaagaaactggcgagcttcgagagcctgccgaaccgtgagcaccacgaagactttatcaagaaatgcgcggaaatcctgattcgtgaggcggtgttcgaaggcaccaaccgtaaaaaccgtgttctgcaatggaacagcccggaggaactgcaaaagctgatggatttcaccctgcgtaccagcccgagcagccatgacgaactgctggatctgctgcgtaacaccgtgaactacagcgttaaaaccggtcacccgtatttcgtgaaccagctgttcagcagcctggacccgtacggtctggtgggtcagtgggcgaccgatgcgctgaacccgagcgtttacacctatgaggtgtctccggtttttaccctgatggaggaagtggttctgcgtgagatgcgtaccattgtgggctttgaaggtggccgtggcgatggtatcttctgcccgggtggcagcattgcgaacggttatgcgatcagctgcgcgcgtcacaagttccagccggaaattaaagcgaccgcgagcgttaccaacctgctgaaaaacatcgcgaacatcattctgctgctgctgcaaaccaaaggtctgcacagcctgccgcgtctggtgctgtttaccagcgaggacgcgcactacagcatcaagaaactgagcagcttcctgggcattggtaccgataacgtttatctgatccgtaccgacgatcgtggtcgtatggacccgagccacctgatccaggagattgagcgtgcgctggcggagggtggcgcgccgtttatggttagcgcgaccgcgggcaccaccgttattggtgcgttcgacccgatcgatcaaattgcggatatctgcgaaaaatacaacctgtggctgcatgtggatgcggcgtggggtggcggtgcgctgatgagcagcaagcaccgtagcctgctgaaaggcatcgagcgtgcggacagcgttacctggaacccgcacaagctgctgaccgcgccgcagcaatgcagcaccctgctgctgcgtcacgagggtctgctgagcgaaacccacagcacccacgcggcgtacctgttccagaaggacaaattttatgataccaagttcgacaccggcaccaagaaattcaacggtgataaacacattcaatgcggccgtcgtgcggacgtgctgaagttctggtttatgtggaaggcgaaaggcaccctgggttttgaaaagcacatcaacaaagttttcgataacgcgaaattctttgcggacagcattcgtaaccgtgtgggttttgagatgctgatcgatcagccggaatgcaccaacgtttgcttctggtacattccggagagcctgcgtaacgcgaagcaagacagcgattataaggaacgtctgcacaaagttgcgccgaagatcaaagagcgtatgatgaaagaaggtagcatgatggtgacctatcaggcgcaaaagagccacccgaacttctttcgtattgtttttcagagcagcggcctggaccgtgcggatatgctgcacctgatcgaggagttcgagcgtctgggtcgtgacctgtaatgataagaattc

SEQ ID NO: 21 - DNA, 1626 bp, Tenebrio molitor
atgccggcgcgtggcgagcaggatgatgtggttcaagacatcattgaggaaccggcgacctacggtgatgcgatcctgagcagcgacgatgaggtgtataccaagttcagcgaacgtccgctgacccaattttaccagccgagccaaaaacgtgcgagcttcgagagcctgccgaaccgtgaacgtcacgaggaatttatccgtaagagcgtggagattctgctgaaagacgcggttttcgaaggcaccagccgtaacaaccgtgttctgcaatggacctgcccggaggaactgagccgtctgatggagtttggtctgaagaacggcccgagcacccacgaggaactgctggaaatcctgaagaaagtggttaactacagcgtgaaaaccggccacccgtatttcgttaaccagctgtttagcagcctggacccgtatggtctggttgcgcaatgggcgaccgatgcgctgaacccgagcgtgtacacctatgaggtgtctccggttttcattctgatggaggaagtggttctgaaggagatgcgtagcatcgtgggtttcgaagcgggccgtggtgatggcatcttctgcccgggtggcagcattgcgaacggttacgcgattagctgcgcgcgttataaattccagccggacatcaagcgtaaaggtctgcacagcctgccgcgtctggttctgttcaccagcgaagatgcgcactatagcattaagaaactgagcagctttctgggtatcggcaccgacaacgtttacctgattcgtaccgacgatcgtggtcgtatggatgtgacccacctgatcggccaaattgaacgtagcctgagcgagggtgcggcgccgttcatggttagcgcgaccgcgggtaccaccgttattggtgcgtttgacccgctgaacgagattgcgagcgtgtgcgaaaagtacaaactgtggctgcatgttgatgcggcgtggggtggcggtgcgctggttagcggcaagcacaaaagcctgctgaagggcatcgagcgtgcggacagcgtgacctggaacccgcacaaactgctgaccgcgccgcagcaatgcagcaccctgctgctgcgtcacgaaggtattctggctgcggcgcacagcaccaacgcggcgtacctgttccagaaggacaaaagctatgataccaagtttgacaccggtgataaacacatccaatgcggccgtcgtgcggatgtgctgaagttctggtttatgtggaaggcgaaaggtaccagcggcctggagaagcacattaacaaagttttcgaaaacgcggcgtattttgcggacagcatccgtaaccgtgagggcttcgaaatggtgattgatcagccggagtgcaccaacgtttgcttttggtatatcccggaaagcctgcgtagctgcaaacaagacagcgattacaaggagcgtctgcacaaagttgcgccgaagattaaagagcgtatgatgaaggaaggtagcatgatggttacctaccaggcgcaaaaacagcacccgaacttctttcgtatcgtgttccagaacagcggcctggacaaggcggatatgatccactttgttgaggaaattgaacgtctgggtaaagacctgtaa

SEQ ID NO: 22 - DNA, 1644 bp, Cryptotermes secundus
atgccggcgagcagcggtatcattaccctgacccagagcctggagaacctgaacggcaagcacggcatcagcggtagctacgaagacatgaccgcgggcgtgaacgtggcggtgccgagcctgagcccgagcccgggttatgttaccgagaagaaaagcacccgtagcgtggcgtggtttgcgagcctgccggaccgtcagcgtcacagccaatttctgaaagaggcggttgacctgatgctggataaagcggtgttcgatgcggcgagccgtaccaaccgtgtggttgaatggcgtagcccggaggaactgaagaaactgattgacctggatctgccggcggaccgtgtgagccacgatcgtctgctgcaactgctgaaggacatcattcaatacagcgttaaaaccggccacccgtatttcgtgaaccagctgtttagcagcgttgacccgtacggcctggtgggtcaatggctgggtgatgcgctgaacccgagcgtttacacctatgaggtttctccggtgtttaccctgatggaggaaaccgttctgtgcgagatgcgtcgtattgtgggcttcccggaaggccgtggtgatggcatcttttgcccgggtggcagcattgcgaacggttacgcgatcagctgcgcgcgttataacttcgttccggacgtgaagaaacgtggcctgcacggtctgccgcgtctggtgctgtttaccagcgaagatgcgcactacagcattaagaaaatggcgagcctgctgggtctgggcagcgacaacgtttatctgatccactgcaacagcaagggcaaaatggatgtgcagcacctggagcaggaaattcagcgtgcgctggaggaaggtgctgcgccgttcatggttagcgcgaccgcgggcaccaccgtgctgggtgcgtttgacccgatcccgaagattgcggacatttgcagcaagtacaaaatgtggctgcacgttgatgcggcgtggggtggcggtgcgctggttagcaagaaacacaagcacctgctggagggcatcgaaaaagcggatagcgtgacctggaacccgcacaaactgctgaccgcgccgcagcaatgcagcaccttcctgctgcgtcacgagggtgttctgagcgcgtgccacagcgcgagcgcgcagtacctgttccaaaaggacaaattttacgatacccagtatgacaccggcgataagcacattcaatgcggtcgtcgtgcggacgtgctgaaattctggtttatgtggaaggcgaaaggcaccgttggtctggaggaacacattgacaccgtgttcgataacgcggcgtactttaccaagcagatcaagaaacgtgaaggcttccgtatggttctgcaagagccggaatgcaccaacgtgtgcttttggtatatcccgccgagcctgcgtggtcacgaggaccagagcgatttcagcgaacgtctgcacaaagttgcgccgcgtattaaggagcgtatgatcaaagaaggtagcatgatggttacctaccagccgctgcgtgaccaaccgaacttctttcgtctggtgctgcaaaacagcggcctggattgggcggacatggattatttcgttcaagagtttgaacgtctgggtagcgatctgtaa

SEQ ID NO: 23 - DNA, 1728 bp, Drosophila melanogaster
atgctggcgagcgaaaactttccgacccaccacttcaaggagagcatctttaaaccgtatagcaccaccagcggtgatgatctggcgagcgtgagcccgctgaccgcgaccgcggcgctggttgcgagcaccagcagcccggcggatagcaccagcaccgtggcgtttgaacaggcgagcaagatgctggcgaacgcggcgaacaataataacaacaacaacaacaacatcaccagcaccaaagacgatctgagcagctttgttgcgagccacccggcggcggagtttgaaggtttcattcgtgcgtgcgtggacgagatcattaagctggcggttttccaaggtaccaaccgtagcagcaaagtggttgagtggcacgaaccggcggagctgcgtcagctgttcgactttcagctgcgtgaacaaggcgagagccaggataagctgcgtgaactgctgcgtgagaccatccgttttagcgtgaaaaccggtcacccgtacttcattaaccaactgtatagcggcgtggacccgtacgcgctggttggtcagtggctgaccgatgcgctgaacccgagcgtgtacacctatgaagttgcgccgctgttcaccctgatggaggaacaagtgctggcggagatgcgtcgtatcgttggttttccgaacggtggtcagggcgacggtattttctgcccgggtggcagcatcgcgaacggctatgcgattagctgcgcgcgttaccgtcacagcccggaaagcaagaaaaacggtctgtttaacgcgaagccgctgatcattttcaccagcgaagacgcgcactacagcgtggagaaactggcgatgtttatgggcttcggtagcgatcacgtgcgtaagatcgcgaccaacgaggttggcaaaatgcgtctgagcgacctggaaaagcaagttaaactgtgcctggagaacggttggcaaccgctgatggttagcgcgaccgcgggcaccaccgttctgggtgcgtttgacgatctggcgggcatcagcgaagtgtgcaagaaatataacatgtggatgcatgttgatgcggcgtggggtggcggtgcgctgatgagcaagaaataccgtcacctgctgaacggtattgagcgtgcggacagcgtgacctggaacccgcacaagctgctggcggcgagccagcaatgcagcaccttcctgacccgtcaccagcaagttctggcgcaatgccacagcaccaacgcgacctacctgttccagaaggacaaattttacgataccagcttcgacaccggcgataagcacatccaatgcggtcgtcgtgcggatgtgttcaaattttggttcatgtggaaggcgaaaggcacccagggtctggaagcgcacgtggagaaggtttttcgtatggcggagttcttcaccgcgaaagtgcgtgaacgtccgggctttgagctggttctggaaagcccggagtgcaccaacattagcttctggtatgttccgccgggtctgcgtgagatggaacgtaaccgtgagttctacgaccgtctgcacaaagtggcgccgaaggttaaagagggcatgatcaagaaaggtagcatgatgattacctatcaaccgctgcgtcagctgccgaacttctttcgtctggtgctgcaaaacagctgcctggaggaaagcgacatggtttacttcctggatgagattgaaagcctggcgcagaacctgtaa

SEQ ID NO: 24 - DNA, 1692 bp, Culex quinquefasciatus
atgccgaccaacggcatgttcgacgtggcgctgcaagttattgaggatgcgaacctgagcagcggcagcgacagcgcgggtgtgagcgaggacgaagatgttcaactgttttgcaccaccggtaacgtggttagcagcaaaccgctgaagaaaccgagcctgaagccggtgaccaccgttaaagacgaagatcagaacaagatgaaaaccaacgcgaagcgttacgcgagcctgccgaaccgtgagcagcaccaacgtttcctgaccgactttctgagcgaagtgctgaacaacgcgatcttcaacgcgaccgatcgtagcaacaaagtgctgaactgggttgacccggaggaactgaagcgtagcattgatctgagcctgaaagcggagccggacagcgatgagaagctgctggaactggcgcgtgcgaccatcgaccacagcgttaagaccggccacccgtacttcatgaaccagctgtttagcagcgtggacgtttatggcttcgcgggtcaatgcctgaccgatgcgctgaacccgagcgtgtacaccttcgaagttagcccggtgtttgttctgatggaggaagtggttctgaaagaaatgcgtaccattgtgggtttcccgggtggcgttggcgacggtatcttttgcccgggtggcagcatggcgaacggctatgcgattagctgcgcgcgttttaagcacatgccggacgtgaagaccaaaggtctgcacagcctgccgcgtctggttattttcaccagcgaagatgcgcactacagcatcaagaaactggcgagctttatgggcatcggtagcgataacgtgtatccgattcgtaccgacgcggttggcaaaatccagccggatcacctggaggcggaaattctgcgtgcgaagagcgagggtgcgctgccgtttatggttagcgcgaccgcgggcaccaccgttattggtgcgtttgacccgctggaacagatcgcggatctgtgccaaaaatacaacctgtggatgcatgtggatgcggcgtggggtggcggtgcgctgatgagcaagaaatatcgtaccctgctgaaaggtgtggagcgtgcggatagcgttacctggaacccgcacaagctgctggcggcgccgcagcaatgcagcaccttcctgacccgtcacgaaggcattctgagcggttgccacagcaccaacgcgacctacctgttccagaaggacaaattttacgatacccaatatgacaccggcgataagcacattcagtgcggtcgtcgtgcggacgttctgaaattctggtttatgtggcgtgcgaagggcaccagcggtttcgagcaacacatcgataaagtgttcgagaacgcggaatactttaccaacagcattaaggcgcgtccgggtttcgaaatggttatcgagaacccggaatgcaccaacgtgtgcttttggtatgttccgccgggtctgcgtcaagtgccgcgtgacagcgcggagtttggtgaacgtctgcacaaagtggcgccgaaggttaaagagcgtatgatgcgtgaaggcagcatgatgatcacctaccagccgattcacgataaaccgaacttctttcgtctggttctgcaaaacagcggtctggacaagagcgatatgaactatatcattgacgagatcgaacgtctggcgagcgatctgtaa

SEQ ID NO: 25 - DNA, 1683 bp, Aedes albopictus
atgccggcgaacggcatgttcgatgtggcgctgcaagttatcgacgatagcaacgtgagcagcggtagcgacagcgcgggcgtgagcgaggatgaagatgttcaactgttttgctcgatgggtaacaccatcgcgccgaaaccgctgaagaaaagcattaccaagaccaaagatgaggaatttagcaagaccgcgaaagcgaacgagaagcgttacgcgagcctgccgaaccgtgaacagcaccagcaattcctgaccgactttctgagcgaggtgctgaacaacgcggttttcaacgcgaccgaacgtgcgaacaaagtgctgaactgggttgatccggagcaactgaagcgtaccctggacctggagctgaaagacgaaccggatagccacgagaagctgctggaactgacccgtgcgaccatcaagcacagcgtgaaaaccggtcacccgtacttcatgaaccagctgtttagcagcgttgatccgtatggttttgcgggccaaattctgaccgacgcgctgaacccgagcgtgtacaccttcgaagttagcccggtgtttgttctgatggaggaagtggttctgaaagaaatgcgtaccattgtgggttacccggatggtgcgggtgatggcattttctgcccgggtggcagcatggcgaacggttatagcatcagctgcgcgcgttttaagcacatgccggatgttaagaccaaaggcctgcacagcctgccgcgtctggtgattttcaccagcgaggacgcgcactacagcgttaagaaactggcgagctttatgggtatcggcagcgacaacgtgtatccgattcgtaccgatgcgatcggtaaaattcgtgttgaccacctggagagcgaaattctgcgtgcgaaagcggagggtgcggtgccgttcatggttagcgcgaccgcgggtaccaccgtgattggtgcgtttgacccgctggaacagattgcggatctgtgcaagaaatacaacctgtggatgcatgttgatgcggcgtggggtggcggtgcgctgatgagcaagaaatatcgtagcctgctgaaaggtatcgaacgtagcgacagcgttacctggaacccgcacaagctgctggcggcgccgcagcaatgcagcaccttcctgacccgtcacgagggcattctgagcgaatgccacagcaccaacgcgacctacctgttccagaaggacaaattttacgatacccaatatgacaccggtgataaacacatccagtgcggccgtcgtgcggacgtgctgaaattctggtttatgtggcgtgcgaagggtaccagcggtctggagcaacacatcgataaagttttcgagaacgcggaacactttaccaacagcattaaggcgcgtgacggtttcgaaatggtggttgagaccccggaatgcaccaacgtgtgcttttggtatgttccgccgggtctgcgtagcgtgccgcgtgatagcgcggagttcaccgaacgtctgcacaaggtggcgccgaaggttaaagagcgtatgatgcgtgaaggtagcatgatgatcacctaccagccgattcacgacaaaccgaacttctttcgtctggttctgcaaaacagcgcgctggacaagagcgatatgaactatatcattgatgagatcgaacgtctggcggcggacctgtaa

SEQ ID NO: 26 - DNA, 1689 bp, Aedes aegypti
atgccggcgaacggcatgttcgatgtggcgctgcaagttatcgacgatagcaacgtgagcagcggtagcgacagcgcgggcgtgagcgaggatgaagatgttcaactgttttgctcgaagggcaacaccattgttccgaaaccgctgaagaaaagcatcagcaagattaaagatgaggaatttagcaagaccgcgaaagcgaacgagaaacgttacgcgagcctgccgagccgtgaacaccaccagcaattcctgaccgactttctgagcgaggtgctgaacaacgcggttttcaacgcgaccgaacgtgcgaacaaggtgctgaactgggttgatccggagcagctgaagcgtaccctggacctggagctgaaagacgaaccggatagccacgagaagctgctggaactgacccgtgcgaccatcaagcacagcgtgaaaaccggtcacccgtacttcatgaaccagctgtttagcagcgttgatccgtatggttttgcgggccaaattctgaccgacgcgctgaacccgagcgtgtacaccttcgaagttagcccggtgtttgttctgatggaggaagtggttctgaaagaaatgcgtaccatcgtgggttacccggacggcaccggtgatggcattttctgcccgggtggcagcatggcgaacggttatagcatcagctgcgcgcgttttaagcacatgccggatgttaagaccaaaggcctgcacagcctgccgcgtctggtgattttcaccagcgaagacgcgcactacagcgttaagaaactggcgagctttatgggtatcggcagcgacaacgtgtatccgattcgtaccgatgcgatcggtaaaattcgtgttgaccacctggagagcgaaattctgcgtgcgaagagcgagggtgcggtgccgttcatggttagcgcgaccgcgggtaccaccgtgattggtgcgtttgacccgctggaacagattgcggatctgtgcaagaaatacaacctgtggatgcatgttgatgcggcgtggggtggcggtgcgctgatgagcaagaaatatcgtagcctgctgaaaggtatcgagcgtagcgacagcgtgacctggaacccgcacaagctgctggcggcgccgcagcaatgcagcaccttcctgacccgtcacgagggcattctgagcgaatgccacagcaccaacgcgacctacctgttccagaaggacaaattttacgatacccaatatgacaccggtgataaacacatccagtgcggccgtcgtgcggatgtgctgaaattctggtttatgtggcgtgcgaagggtaccagcggcctggaacaacacatcgacaaagttttcgagaacgcggaacactttaccagcagcattaaggcgcgtgagggtttcgaaatggtggttgagaacccggaatgcaccaacgtgtgcttttggtatgttccgccgggtctgcgtaacgtgccgcgtgatagcgcggagttcaccgaacgtctgcacaaagtggcgccgaaggttaaagagcgtatgatgcgtgaaggtagcatgatgatcacctaccagccgattcacgacaaaccgaacttctttcgtctggttctgcaaaacagcgcgctggacaagagcgatatgaactatatcattgatgagatcgaacgtctggcggcggacctgaagccgtaa

SEQ ID NO: 27 - DNA, 1719 bp, Anopheles sinensis
atgccggcgaacggtgtgaacagcgttgagctggaagtgatcgaggatgttgcgaccacctacgcgagcggtagcgacagcgcgggcgtgagcgaggatgaagatgtgcagcaactgttcgttagcggtgcgcaccacattagcagcgtgccgccgctgaagaaagcggttgagacccgtggcaagggtacccagctgcaaggtccggcgagcgagggtgcggcggcggcggaagtgagcgaaaaacgttatgcgagcctgccgaaccgtgagcagcaccagcaattcctgaccgattttctgaccgaagtgctgaacagcgcggttttcaacgcgaccgatcgtgcgaacaaggtgctgaactgggttgacccggaggaactgaagcgtaccctggacctggcgatcaaacaagagccggatacccacgagaagctgctggaactgacccgtgcgaccattcgtcacagcgtgaaaaccggtcacccgtacttcatgaaccagctgttcagcagcgtggacccgtacggttttgcgggccaagttctgaccgacgcgctgaacccgagcgtgtacaccttcgaagttagcccggtgtttgttctgatggaggaagtggttctgcgtgagatgcgtaccatcgtgggttatccgaacggcgaaggtgacggcattttcgcgccgggtggcagcatggcgaacggttacgcgatcagctgcgcgcgttataagtttatgccggatgttaaagcgaaaggtctgcatgcgctgccgcgtctggtgattttcaccagcgaagacgcgcactacagcgttaagaaactggcgagctttatgggtatcggcagcgacaacgtgtatgcgattaagaccgatgcgatcggtaaaatttgcgttgaccacctggagagcgaaatcctgcgtgcgaagcaggaaggtgcgctgccgttcatggttagcgcgaccgcgggtaccaccgttattggtgcgtttgacccgctggaacaaattgcggatctgtgcgcgaaatacaacctgtggatgcatgtggatgcggcgtggggtggcggtgcgctgatgagcaagaaatatcgtaccctgctgaaaggtatcgagcgtagcgacagcgttacctggaacccgcacaagctgctggcggcgccgcagcaatgcagcaccctgctgacccgtcaccgtaacattctgagcgaatgccacagcaccaacgcgacctacctgttccagaaggacaaattttacgatacccgttatgacaccggtgataaacacatccaatgcggccgtcgtgcggatgttctgaaattctggtttatgtggcgtgcgaagggtaccgcgggctttgagcagcacattgacaaagtgttcgagaacgcggaacactttaccagcagcatcaaggcgcgtccgggtttcgaaatggttattgagaacccggaatgcaccaacgtgtgcttttggtatgttccgccgggtctgcgtagcgtgccgcgtgatagcgcggagtttcgtgaacgtctgcacaaagtggcgccgaaggttaaagagcgtatgatgaaggaaggtagcatgatgatcacctaccagccgattcacgacaaaccgaacttctttcgtctggttctgcaaaacagcagcctggacaagagcgatatgaactatatcattgatgagatcgaacgtctgggcaaagacctgtaa

SEQ ID NO: 28 - protein, 15 aa, Artificial Sequence
  Conserved N-Term 15-aa mosquito ADC sequence
SGSDSAGVSEDEDVQ

SEQ ID NO: 29 - protein, 491 aa, Artificial Sequence
  Consensus Sequence
  MISC_FEATURE (4)..(4): X is N, S, I, V, or D
  MISC_FEATURE (5)..(5): X is R or Q
  MISC_FEATURE (6)..(6): X is E, S, A, R, K, or absent
  MISC_FEATURE (7)..(7): X is Q, H, V, S, M, L, or R
  MISC_FEATURE (9)..(9): X is Q, E, or F
  MISC_FEATURE (10)..(10): X is R, Q, T, K, or E
  MISC_FEATURE (12)..(12): X is L or I
  MISC_FEATURE (13)..(13): X is T, R, or K
  MISC_FEATURE (14)..(14): X is D, E, Q, N, K, or S
  MISC_FEATURE (15)..(15): X is F, C, A, M, L, S, or A
  MISC_FEATURE (16)..(16): X is L, I, F, V, or M
  MISC_FEATURE (17)..(17): X is S, T, E, or D
  MISC_FEATURE (18)..(18): X is E, V, N, S, I, or L
  MISC_FEATURE (19)..(19): X is V, I, L, or M
  MISC_FEATURE (21)..(21): X is N, S, K, Q, or E
  MISC_FEATURE (22)..(22): X is G, N, S, A, K, E, or D
  MISC_FEATURE (24)..(24): X is I or V
  MISC_FEATURE (26)..(26): X is N, S, E, D, or Q
  MISC_FEATURE (27)..(27): X is A, G, or D
  MISC_FEATURE (29)..(29): X is D, E, K, R, N, or S
  MISC_FEATURE (31)..(31): X is S, A, K, E, or N
  MISC_FEATURE (33)..(33): X is K, R, P, or L
  MISC_FEATURE (35)..(35): X is L or V
  MISC_FEATURE (36)..(36): X is N, E, D, or Q
  MISC_FEATURE (37)..(37): X is W or F
  MISC_FEATURE (38)..(38): X is V, K, R, H, or T
  MISC_FEATURE (39)..(39): X is D, N, Q, E, C, or A
  MISC_FEATURE (41)..(41): X is E, K, or N
  MISC_FEATURE (42)..(42): X is E, Q, I, or V
  MISC_FEATURE (44)..(44): X is K, Q, S, E, A, or R
  MISC_FEATURE (45)..(45): X is R, N, E, K, or S
  MISC_FEATURE (46)..(46): X is S, T, I, R, L, or K
  MISC_FEATURE (47)..(47): X is I, L, F, V, or M
  MISC_FEATURE (48)..(48): X is D, N, or E
  MISC_FEATURE (49)..(49): X is L, F, or M
  MISC_FEATURE (50)..(50): X is S, E, A, K, G, or D
  MISC_FEATURE (51)..(51): X is L, I, or V
  MISC_FEATURE (52)..(52): X is K, R, or S
  MISC_FEATURE (53)..(53): X is D, A, Q, S, T, G, or N
  MISC_FEATURE (54)..(54): X is E, N, T, D, A, or G
  MISC_FEATURE (55)..(55): X is P, S, A, or R
  MISC_FEATURE (56)..(56): X is D, S, or V
  MISC_FEATURE (57)..(57): X is S or T
  MISC_FEATURE (58)..(58): X is D or H
  MISC_FEATURE (59)..(59): X is E, Q, or D
  MISC_FEATURE (60)..(60): X is K, R, E, T, or D
  MISC_FEATURE (62)..(62): X is L or M
  MISC_FEATURE (63)..(63): X is E, Q, L, or K
  MISC_FEATURE (64)..(64): X is L, T, V, or I
  MISC_FEATURE (65)..(65): X is A, T, L, I, or V
  MISC_FEATURE (66)..(66): X is R, K, L, D, or N
  MISC_FEATURE (67)..(67): X is A, D, E, or K
  MISC_FEATURE (68)..(68): X is T, M, V, or I
  MISC_FEATURE (69)..(69): X is I or V
  MISC_FEATURE (70)..(70): X is D, K, R, T, N, or H
  MISC_FEATURE (71)..(71): X is H or Y
  MISC_FEATURE (79)..(79): X is Y or F
  MISC_FEATURE (81)..(81): X is M or V
  MISC_FEATURE (87)..(87): X is S, G, C, or A
  MISC_FEATURE (88)..(88): X is V or L
  MISC_FEATURE (90)..(90): X is P or V
  MISC_FEATURE (92)..(92): X is G or A
  MISC_FEATURE (93)..(93): X is F or L
  MISC_FEATURE (94)..(94): X is A, I, or V
  MISC_FEATURE (95)..(95): X is G or A
  MISC_FEATURE (97)..(97): X is V, C, I, W, or L
  MISC_FEATURE (98)..(98): X is L, I, or A
  MISC_FEATURE (99)..(99): X is T or G
  MISC_FEATURE (105)..(105): X is S or T
  MISC_FEATURE (109)..(109): X is F or Y
  MISC_FEATURE (114)..(114): X is V or C
  MISC_FEATURE (116)..(116): X is V, T, or I
  MISC_FEATURE (121)..(121): X is V, T, I, E, or A
  MISC_FEATURE (124)..(124): X is K, R, or E
  MISC_FEATURE (128)..(128): X is T, A, S, K, or H
  MISC_FEATURE (129)..(129): X is I or L
  MISC_FEATURE (132)..(132): X is F, Y, or W
  MISC_FEATURE (133)..(133): X is P, E, S, H, or K
  MISC_FEATURE (134)..(134): X is G, D, N, or A
  MISC_FEATURE (135)..(135): X is absent or D
  MISC_FEATURE (136)..(136): X is G or A
  MISC_FEATURE (137)..(137): X is V, S, A, T, E, H, N, I, K, or R
  MISC_FEATURE (140)..(140): X is G or A
  MISC_FEATURE (141)..(141): X is I or L
  MISC_FEATURE (143)..(143): X is C, A, V, or S
  MISC_FEATURE (148)..(148): X is M, I, or T
  MISC_FEATURE (149)..(149): X is A or S
  MISC_FEATURE (153)..(153): X is A, S, or G
  MISC_FEATURE (157)..(157): X is A or G
  MISC_FEATURE (159)..(159): X is F, Y, or H
  MISC_FEATURE (160)..(160): X is K, H, R, or N
  MISC_FEATURE (161)..(161): X is H, R, F, Y, V, or A
  MISC_FEATURE (162)..(162): X is M, C, A, Y, I, or Q
  MISC_FEATURE (164)..(164): X is D or E
  MISC_FEATURE (165)..(165): X is V, I, A, or M
  MISC_FEATURE (167)..(167): X is T, A, S, N, K, or R
  MISC_FEATURE (171)..(171): X is H, C, or Q
  MISC_FEATURE (172)..(172): X is S, A, N, G, or C
  MISC_FEATURE (173)..(173): X is L, G, or I
  MISC_FEATURE (178)..(178): X is I, M, or L
  MISC_FEATURE (182)..(182): X is E, A, I, or Q
  MISC_FEATURE (184)..(184): X is A or S
  MISC_FEATURE (188)..(188): X is I, V, or S
  MISC_FEATURE (188)..(189): X is I, V, Q, or G
  MISC_FEATURE (191)..(191): X is L or M
  MISC_FEATURE (192)..(192): X is A or S
  MISC_FEATURE (194)..(194): X is F or L
  MISC_FEATURE (195)..(195): X is M, L, Q, or E
  MISC_FEATURE (197)..(197): X is I or L
  MISC_FEATURE (199)..(199): X is S, M, T, or N
  MISC_FEATURE (204)..(204): X is P, A, K, or L
  MISC_FEATURE (205)..(205): X is I or V
  MISC_FEATURE (206)..(206): X is R, H, K, Q, E, N, or C
  MISC_FEATURE (207)..(207): X is T or C
  MISC_FEATURE (208)..(208): X is D, C, or N
  MISC_FEATURE (209)..(209): X is A, S, N, E, P, M, or D
  MISC_FEATURE (210)..(210): X is V, I, R, L, or K
  MISC_FEATURE (212)..(212): X is K or R
  MISC_FEATURE (213)..(213): X is I, L , or M
  MISC_FEATURE (214)..(214): X is Q, R, C, I, or D
  MISC_FEATURE (215)..(215): X is P, V, I, or M
  MISC_FEATURE (216)..(216): X is D, E, K, N, G, S, T, or Q
  MISC_FEATURE (217)..(217): X is H or N
  MISC_FEATURE (219)..(219): X is E, V, or I
  MISC_FEATURE (220)..(220): X is A, S, T, E, M, G, or Q
  MISC_FEATURE (221)..(221): X is E or Q
  MISC_FEATURE (222)..(222): X is I or V
  MISC_FEATURE (223)..(223): X is L, E, I, or Q
  MISC_FEATURE (225)..(225): X is A, V, or S
  MISC_FEATURE (226)..(226): X is K or L
  MISC_FEATURE (227)..(227): X is S, A, Q, L, T, N, E, or R
  MISC_FEATURE (230)..(230): X is absent or N
  MISC_FEATURE (232)..(232): X is V, L, S, I, or A
  MISC_FEATURE (235)..(235): X is M or L
  MISC_FEATURE (237)..(237): X is S or V
  MISC_FEATURE (240)..(240): X is A or S
  MISC_FEATURE (245)..(245): X is I or L
  MISC_FEATURE (251)..(251): X is L or I
  MISC_FEATURE (252)..(252): X is E, D, N, or P
  MISC_FEATURE (253)..(253): X is Q, D, K, or E
  MISC_FEATURE (254)..(254): X is I or V
  MISC_FEATURE (256)..(256): X is D, E, or S
  MISC_FEATURE (257)..(257): X is L, I, or V
  MISC_FEATURE (259)..(259): X is Q, K, A, E, or S
  MISC_FEATURE (261)..(261): X is Y or H
  MISC_FEATURE (262)..(262): X is N, D, Q, G, or K
  MISC_FEATURE (263)..(263): X is L, M, or I
  MISC_FEATURE (265)..(265): X is M, F, or L
  MISC_FEATURE (267)..(267): X is V or I
  MISC_FEATURE (275)..(275): X is A or V
  MISC_FEATURE (276)..(276): X is L or M
  MISC_FEATURE (277)..(277): X is M or V
  MISC_FEATURE (279)..(279): X is K, Q, P, R, A, or G
  MISC_FEATURE (281)..(281): X is Y or H
  MISC_FEATURE (282)..(282): X is R or K
  MISC_FEATURE (283)..(283): X is T, S, H, A, or K
  MISC_FEATURE (285)..(285): X is L or V
  MISC_FEATURE (286)..(286): X is K or E
  MISC_FEATURE (288)..(288): X is V or I
  MISC_FEATURE (291)..(291): X is A or S
  MISC_FEATURE (301)..(301): X is L or M
  MISC_FEATURE (303)..(303): X is A, T, or S
  MISC_FEATURE (304)..(304): X is A, T, or S
  MISC_FEATURE (311)..(311): X is F or L
  MISC_FEATURE (313)..(313): X is T, C, or L
  MISC_FEATURE (315)..(315): X is H or Q
  MISC_FEATURE (316)..(316): X is E, R, P, or Q
  MISC_FEATURE (317)..(317): X is G or N
  MISC_FEATURE (318)..(318): X is I, L, or V
  MISC_FEATURE (320)..(320): X is S, A, or Q
  MISC_FEATURE (321)..(321): X is G, E, P, D, A, or Q
  MISC_FEATURE (322)..(322): X is C or A
  MISC_FEATURE (323)..(323): X is H or N
  MISC_FEATURE (325)..(325): X is T, S, or A
  MISC_FEATURE (326)..(326): X is N or S
  MISC_FEATURE (328)..(328): X is T, A, or Q
  MISC_FEATURE (336)..(336): X is F or S
  MISC_FEATURE (340)..(340): X is Q, R, or K
  MISC_FEATURE (341)..(341): X is Y or F
  MISC_FEATURE (343)..(343): X is T or L
  MISC_FEATURE (354)..(354): X is A or P
  MISC_FEATURE (357)..(357): X is L or Y
  MISC_FEATURE (361)..(361): X is F, L, or Y
  MISC_FEATURE (364)..(364): X is R or K
  MISC_FEATURE (368)..(368): X is T or S
  MISC_FEATURE (369)..(369): X is S, A, T, D, I, K, or N
  MISC_FEATURE (371)..(371): X is L or F
  MISC_FEATURE (372)..(372): X is E, Q, or A
  MISC_FEATURE (373)..(373): X is Q, A, D, or K
  MISC_FEATURE (375)..(375): X is I or V
  MISC_FEATURE (376)..(376): X is D or N
  MISC_FEATURE (377)..(377): X is K, R, S, or T
  MISC_FEATURE (378)..(378): X is V, I, or N
  MISC_FEATURE (380)..(380): X is E, G, or D
  MISC_FEATURE (381)..(381): X is T, N, R, or K
  MISC_FEATURE (382)..(382): X is A or T
  MISC_FEATURE (383)..(383): X is E, K, R, or A
  MISC_FEATURE (384)..(384): X is Y, F, or H
  MISC_FEATURE (385)..(385): X is F or L
  MISC_FEATURE (386)..(386): X is T or A
  MISC_FEATURE (387)..(387): X is N, S, R, Q, T, E, or D
  MISC_FEATURE (388)..(388): X is S, N, Q, L, C, or K
  MISC_FEATURE (389)..(389): X is I, L, or V
  MISC_FEATURE (390)..(390): X is K or R
  MISC_FEATURE (391)..(391): X is A, S, Q, or N
  MISC_FEATURE (392)..(392): X is R, Q, or T
  MISC_FEATURE (393)..(393): X is P, D, E, K, or S
  MISC_FEATURE (394)..(394): X is G or D
  MISC_FEATURE (396)..(396): X is E, V, or R
  MISC_FEATURE (397)..(397): X is M or L
  MISC_FEATURE (399)..(399): X is I, V, L, or absent
  MISC_FEATURE (400)..(400): X is E, D, S, P, A, or W
  MISC_FEATURE (401)..(401): X is N, T, Q, H, E, K, or P
  MISC_FEATURE (404)..(404): X is C or Y
  MISC_FEATURE (407)..(407): X is V or I
  MISC_FEATURE (412)..(412): X is V, I, or L
  MISC_FEATURE (414)..(414): X is P, S, K, or E
  MISC_FEATURE (415)..(415): X is G, S, K, or R
  MISC_FEATURE (416)..(416): X is L or M
  MISC_FEATURE (417)..(417): X is R or Q
  MISC_FEATURE (418)..(418): X is Q, S, N, G, D, or A
  MISC_FEATURE (419)..(419): X is V, M, E, L, A, R, C, or Q
  MISC_FEATURE (420)..(420): X is P, A, K, or E
  MISC_FEATURE (421)..(421): X is R, I, S, D, Q, or V
  MISC_FEATURE (422)..(422): X is D, N, E, or Q
  MISC_FEATURE (423)..(423): X is S, absent, D, or E
  MISC_FEATURE (424)..(424): X is A, S, D, G, absent, P, or H
  MISC_FEATURE (425)..(425): X is E, D, R, or H
  MISC_FEATURE (426)..(426): X is F or Y
  MISC_FEATURE (427)..(427): X is G, T, R, E, K, V, or S
  MISC_FEATURE (428)..(428): X is E, D, G, or Q
  MISC_FEATURE (429)..(429): X is R, K, or E
  MISC_FEATURE (430)..(430): X is L or I
  MISC_FEATURE (432)..(432): X is K or R
  MISC_FEATURE (433)..(433): X is V or I
  MISC_FEATURE (436)..(436): X is K or R
  MISC_FEATURE (437)..(437): X is V or I
  MISC_FEATURE (440)..(440): X is K or H
  MISC_FEATURE (442)..(442): X is M or I
  MISC_FEATURE (443)..(443): X is R or K
  MISC_FEATURE (444)..(444): X is E, D, or N
  MISC_FEATURE (446)..(446): X is S, T, or N
  MISC_FEATURE (449)..(449): X is I, M, or V
  MISC_FEATURE (453)..(453): X is P or A
  MISC_FEATURE (454)..(454): X is I, L, or Q
  MISC_FEATURE (455)..(455): X is H, K, R, or N
  MISC_FEATURE (456)..(456): X is D, E, S, T, L, G, Q, or N
  MISC_FEATURE (457)..(457): X is K, V, H, or Q
  MISC_FEATURE (463)..(463): X is L, F, or I
  MISC_FEATURE (465)..(465): X is L or F
  MISC_FEATURE (467)..(467): X is N or D
  MISC_FEATURE (469)..(469): X is A, G, or S
  MISC_FEATURE (470)..(470): X is L or V
  MISC_FEATURE (471)..(471): X is D, N, T, or E
  MISC_FEATURE (472)..(472): X is K, H, or R
  MISC_FEATURE (473)..(473): X is S, D, G, N, Q, T, or A
  MISC_FEATURE (476)..(476): X is N, L, D, M, T, V, or I
  MISC_FEATURE (477)..(477): X is Y or H
  MISC_FEATURE (478)..(478): X is I, F, or L
  MISC_FEATURE (479)..(479): X is I or V
  MISC_FEATURE (480)..(480): X is D, R, N, E, or Q
  MISC_FEATURE (481)..(481): X is E or T
  MISC_FEATURE (482)..(482): X is I or F
  MISC_FEATURE (484)..(484): X is R or K
  MISC_FEATURE (485)..(485): X is L or Y
  MISC_FEATURE (486)..(486): X is A, G, or S
  MISC_FEATURE (487)..(487): X is S, A, K, C, E, or H
  MISC_FEATURE (488)..(488): X is D, E, or N
  MISC_FEATURE (490)..(490): X is absent or K
  MISC_FEATURE (491)..(491): X is absent or P
SLPXXXXHXXFXXXXXXXXLXXAXFXXTXRXNXVXXXXXPXXLXXXXXXXXXXXXXXXXXLXXXXXXXXXXSVKTGHPXFXNQLFSXXDXYXXXXQXXXDALNPXVYTXEVSPXFXLMEEXVLXEMRXXVGXXXXXXGDXXFXPGGSXXNGYXISCXRXXXXPXXKXKGLXXXPRLVXFTSXDXHYSXXKXXSXXGXGXDNVYXXXXXXXGXXXXXXLXXXXXRXXXEGXAXPFXVXATXGTTVXGAFDPXXXXAXXCXKXXXWXHXDAAWGGGXXXSXKXXXLXXGXERXDSVTWNPHKXLXXPQQCSTXLXRXXXXLXXXXSXXAXYLFQKDKXYDTXXDXGDKHIQCGRRXDVXKFWXMWXAKGXXGXXXHXXXXFXXXXXXXXXXXXXXXFXXVXXXPEXTNXCFWYXPXXXXXXXXXXXXXXXXXHXXAPXXKEXMXXXGXMMXTYQXXXXXPNFFRXVXQXSXXXXXDMXXXXXXXEXXXXXLXX